(19) United States
(12) Patent Application Publication
(10) Pub. No.: US 2026/0297498 A1
(43) Pub. Date: Oct. 1, 2026

(54) Miniaturized, Automated, And Cost-Effective Microfluidic Reactor Device

(71) Applicant: The Regents of The University of Michigan, Ann Arbor, MI (US)

(72) Inventors: Xudong FAN, Saline, MI (US); Majid AALIZADEH, Jackson, MI (US)

(73) Assignee: The Regents of The University of Michigan, Ann Arbor, MI (US)

(21) Appl. No.: 19/633,260

(22) Filed: Mar. 30, 2026

Related U.S. Application Data
(60) Provisional application No. 63/779,599, filed on Mar. 28, 2025.

(51) Int. Cl. C12M 1/36; C12M 1/00; C12M 1/12; C12M 1/32; C12M 1/34; C12M 3/00

(52) U.S. Cl. CPC C12M 41/48; C12M 23/12; C12M 23/42; C12M 23/52; C12M 29/14; C12M 37/04; C12M 41/32

(57) ABSTRACT

An automated microfluidic reactor analyzes at least one sample that reacts with one or more reagents and includes a moveable magazine component with microwells, each having an inlet and a reservoir. At least one microwell is configured to hold one or more reagents. The reactor also has at least one microfluidic connector that has at least one microfluidic channel with an aperture. The at least one microfluidic connector reversibly interfaces with at least one microwell to establish fluid communication therebetween. The reactor also has a first motor for translating (e.g., rotating) the moveable magazine component, a second motor for translating (e.g., linearly) the microfluidic connector. Further, at least one reactor chamber is in fluid communication with the microfluidic channel of the microfluidic connector and is configured to receive a test sample optionally containing one or more analytes. The reactor may further have a pump or pipette device to move fluids.

### CROSS-REFERENCE TO RELATED APPLICATIONS

This application claims the benefit of U.S. Provisional Application No. 63/779,599, filed Mar. 28, 2025. The entire disclosure of the above application is incorporated herein by reference.

### GOVERNMENT SUPPORT

This invention was made with government support under DA052941 awarded by the National Institutes of Health. The government has certain rights in the invention.

### FIELD

The present disclosure relates to a miniaturized, automated, cost-effective microfluidic reactor devices, such as a biosensor or bioreactor, for analyzing a sample to be reacted with one or more reagents.

### BACKGROUND

This section provides background information related to the present disclosure which is not necessarily prior art.

Point-of-care (POC) biosensors are transforming the landscape of healthcare diagnostics by enabling rapid, on-site detection of diseases and biomarkers. Traditionally, diagnostic tests require complex and costly equipment and highly-trained personnel within centralized laboratories, leading to delays in obtaining results. In contrast, POC biosensors allow for immediate diagnostic feedback at or near the site of patient care, providing clinicians with real-time data that can guide timely medical decisions and interventions. Such POC biosensors are compact, user-friendly, and often do not require extensive training, making them ideal for use in emergency rooms, clinics, field hospitals, and even home settings. These benefits not only enhance patient outcomes but also reduce the burden on healthcare systems, by minimizing the need for hospital visits and laboratory infrastructure. Their applications range from glucose monitoring for diabetes patients to detecting infectious diseases such as HIV, malaria, and, more recently, COVID-19, among others.

Enzyme-linked immunosorbent assay (ELISA), one of the most widely used and well-established biosensing techniques, relies on antigen-antibody interactions to detect specific biomolecules. The simplicity, robustness, and specificity of ELISA have made it a cornerstone in clinical diagnostics and laboratory research. Traditionally, immunoassays based on 96-well plates in combination with optical intensity-based detection in signal generation methods, such as colorimetry, fluorescence-based, and chemiluminescence, are widely used for sensitive and quantitative analyte detection in a broad range of applications. Long assay duration and the large dimensions of machines, as well as the high cost per assay, can make them only suitable for laboratory testing.

Further, many widely used commercial ELISA machines have large footprints (large dimensions and heavy), are expensive, or have other shortcomings. For example, most commercial machines operate based on the combination of robotic (mechanical) movement with pipetting. Robotic arm-based ELISA utilizes liquid handling systems for automated pipetting, washing, and detection in microplates, providing high throughput and compatibility with conventional workflows. However, robotic arm ELISA requires significant laboratory space and investment. The prices of these machines are at least tens of thousands of U.S. dollars, leading to an immense need for the development of a cost-effective ELISA machine. To this end, development of POC ELISA-based biosensors would be advantageous.

### SUMMARY

This section provides a general summary of the disclosure, and is not a comprehensive disclosure of its full scope or all of its features.

The present disclosure relates to an automated microfluidic reactor for analyzing a sample to be reacted with one or more reagents. In certain aspects, the reactor comprises a moveable magazine component comprising a plurality of microwells. Each microwell of the plurality of microwells comprises an inlet and a reservoir. At least one of the plurality of microwells is configured to hold one or more reagents. The reactor also comprises at least one microfluidic connector comprising at least one microfluidic channel having an aperture. The at least one microfluidic connector reversibly interfaces with at least one microwell of the plurality of microwells to establish fluid communication therebetween. The reactor further has a first motor for translating the moveable magazine component with respect to the microfluidic connector and a second motor for translating the microfluidic connector from at least a first position where the microfluidic connector contacts the moveable magazine component to a second position where the microfluidic connector is not in contact with the moveable magazine component. At least one of the translating the moveable magazine component or the translating of the at least one microfluidic connector is rotational and the other is linear. The reactor comprises at least one reactor chamber in fluid communication with the microfluidic channel of the microfluidic connector. The at least one reactor chamber is configured to recive a test sample that optionally contains one or more analytes. Further, the reactor includes a pump or a pipette device for moving or transporting fluids from the moveable magazine component through the microfluidic connector to the reactor chamber.

In one aspect, the translating of the moveable magazine component is rotating and the translating of the at least one microfluidic connector is linear.

In one aspect, the pump is for transporting fluids from the moveable magazine component through the microfluidic connector to the at least one reactor chamber.

In one aspect, the pipette is a multi-channel pipette device for transporting fluids from the moveable magazine component through the microfluidic connector to the at least one reactor chamber.

In one aspect, the moveable magazine component has one or more covers or seals that may be removed or punctured to establish fluid communication with one or more of the plurality of microwells.

In one further aspect, the inlet of each microwell of the plurality of microwells has a seal and the at least one microfluidic connector comprises a needle component in fluid communication with the microfluidic channel that is configured to puncture at least a portion of the seal.

In one further aspect, the one or more seals include a single seal used to cover a surface of the moveable magazine component including the plurality of microwells.

In one aspect, the at least one reactor chamber is a plurality of reactor chambers and the at least one microfluidic connector is a multi-channel microfluidic connector comprising a plurality of microfluidic channels in fluid communication with the plurality of reactor chambers.

In one aspect, at least one microwell of the plurality of microwells further comprises a wicking material.

In one aspect, the automated microfluidic reactor further comprises a wash system comprising a washing fluid and a second pump in fluid communication with the microfluidic connector.

In one further aspect, the wash system further comprises a fluid reservoir and the moveable magazine component further comprises at least one wash inlet and at least one wash outlet for transporting the washing fluid from the fluid reservoir to the at least one microfluidic connector.

In one aspect, the at least one microfluidic connector defines a recess in which a microwell of the plurality of microwells reversibly seats. The at least one microfluidic connector comprises a needle component having at least one aperture to provide fluid communication between the recess and the microfluidic channel.

In one aspect, the automated microfluidic reactor further comprises at least one detector for analyzing fluids in the at least one reactor chamber.

In one aspect, the automated microfluidic reactor further comprises a control system for controlling operation of the first motor, the second motor, the pump, and the at least one detector.

In one aspect, at least a portion of the microwells of the plurality of microwells store the one or more reagents.

In one aspect, the test sample comprises one or more target analytes and the one or more reagents are used in one or more reactions to detect the one or more target analytes.

In one further aspect, the one or more reagents are part of an Enzyme Linked ImmunoSorbent Assay (ELISA)-based system.

In one further aspect, the automated microfluidic reactor further comprises one or more detectors for measuring one or more of luminescence, fluorescence, or colorimetry to determine an amount of the one or more target analytes present in the test sample.

In one further aspect, the automated microfluidic reactor further comprises at least one processor configured to receive output from one or more detectors and configured to determine an amount of the one or more target analytes present in the test sample.

In one aspect, the at least one microfluidic connector is a plurality of microfluidic connectors each configured to engage independent microwells of the plurality of microwells in the moveable magazine component. Each of the plurality of microfluidic connectors is in fluid communication with the at least one reactor chamber.

In one aspect, the at least one reactor chamber comprises a plurality of reactor chambers and each of the plurality of microfluidic connectors is in fluid communication with a distinct reactor chamber of the plurality of reactor chambers.

In one aspect, the at least one microfluidic connector is configured to engage at least one microwell of the plurality of microwells in the moveable magazine component. The at least one microfluidic channel comprises a plurality of microfluidic channels and the at least one reactor chamber comprises a plurality of reactor chambers each in fluid communication with a microfluidic channel of the plurality of microfluidic channels.

In one further aspect, each of the plurality of microfluidic channels is in fluid communication with a distinct reactor chamber of the plurality of reactor chambers.

In one aspect, each microwell of the plurality of microwells has a respective volume of less than or equal to about 1 milliliter (mL).

In one aspect, a maximum dimension of the automated microfluidic reactor is less than or equal to about 25 cm and the automated microfluidic reactor is portable.

In one aspect, the moveable magazine component is a disk component to be rotated and the at least one microfluidic connector is configured to translate linearly. The first motor rotates the moveable magazine component with respect to the microfluidic connector. The second motor linearly translates the at least one microfluidic connector from the first position to the second position.

In certain further aspects, the present disclosure relates to an automated microfluidic reactor for analyzing a sample to be reacted with one or more reagents. The reactor may comprise a moveable magazine component comprising a plurality of microwells. Each microwell of the plurality of microwells comprises an inlet, a reservoir, and an outlet. At least one of the plurality of microwells is configured to receive a test sample and at least one of the plurality of microwells is configured to hold one or more reagents. The reactor may further comprise at least one microfluidic connector comprising at least one microfluidic channel and a recess in which a microwell of the plurality of microwells reversibly seats. The at least one microfluidic connector comprises a needle component having at least one aperture to provide fluid communication between the recess and the microfluidic channel. A first motor is incorporated for translating the moveable magazine component with respect to the microfluidic connector. A second motor is incorporated for translating the microfluidic connector from at least a first position where the microfluidic connector contacts the moveable magazine component to a second position where the microfluidic connector is not in contact with the moveable magazine component. At least one reactor chamber is provided in fluid communication with the microfluidic channel of the microfluidic connector. The reactor further includes a pump for transporting fluids from the moveable magazine component through the microfluidic connector to the reactor chamber.

In one aspect, the automated microfluidic reactor further comprises at least one detector for analyzing fluids in the at least one reactor chamber.

In one further aspect, the automated microfluidic reactor further comprises a control system for controlling operation of the first motor, the second motor, the pump, and the at least one detector.

In one aspect, the automated microfluidic reactor further comprises a wash system comprising a washing fluid and a second pump in fluid communication with the microfluidic connector.

In one further aspect, the wash system further comprises a fluid reservoir and the moveable magazine component further comprises at least one wash inlet and at least one wash outlet for transporting the washing fluid from the fluid reservoir to the at least one microfluidic connector.

In one aspect, the outlet of each microwell of the plurality of microwells has a seal and the needle component is configured to puncture at least a portion of the seal.

In one aspect, at least a portion of the microwells of the plurality of microwells store the one or more reagents.

In one aspect, the test sample comprises one or more target analytes and the one or more reagents are used in one or more reactions to detect the one or more target analytes.

In one further aspect, the one or more reagents are part of an Enzyme Linked ImmunoSorbent Assay (ELISA)-based system.

In one further aspect, the automated microfluidic reactor further comprises one or more detectors for measuring one or more of luminescence, fluorescence, or colorimetry to determine an amount of the one or more target analytes present in the test sample.

In one further aspect, the automated microfluidic reactor further comprises at least one processor configured to receive output from one or more detectors and configured to determine an amount of the one or more target analytes present in the test sample.

In one aspect, the inlet of at least one microwell of the plurality of microwells has a removable cover.

In one aspect, the at least one microfluidic connector comprises a plurality of microfluidic connectors, wherein each of the plurality of microfluidic connectors is configured to engage independent microwells of the plurality of microwells in the moveable magazine component, so that each independent microwell seats in the recess of a respective microfluidic connector of the plurality of microfluidic connectors. Each of the plurality of microfluidic connectors is in fluid communication with the at least one reactor chamber.

In one aspect, the at least one reactor chamber comprises a plurality of reactor chambers and the at least one microfluidic channel is a plurality of microfluidic channels, so that each of the plurality of microfluidic channels is in fluid communication with a distinct reactor chamber of the plurality of reactor chambers.

In one aspect, the at least one microfluidic connector is configured to engage at least one microwell of the plurality of microwells in the moveable magazine component. The at least one microfluidic channel comprises a plurality of microfluidic channels and the at least one reactor chamber comprises a plurality of reactor chambers each in fluid communication with a microfluidic channel of the plurality of microfluidic channels.

In one further aspect, the at least one reactor chamber comprises a plurality of reactor chambers and each of the plurality of microfluidic connectors is in fluid communication with a distinct reactor chamber of the plurality of reactor chambers.

In one aspect, the automated microfluidic reactor further comprises at least one processor to control operation of at least one of the first motor, the second motor, or the pump or pipette device.

In one aspect, each microwell of the plurality of microwells has a respective volume of less than or equal to about 1 milliliter (mL).

In one aspect, a maximum dimension of the automated microfluidic reactor is less than or equal to about 25 cm and the automated microfluidic reactor is portable.

In one aspect, the moveable magazine component is a disk component to be rotated. The at least one microfluidic connector is configured to translate linearly, the first motor rotates the moveable magazine component with respect to the microfluidic connector, the second motor linearly translated the at least one microfluidic connector from the first position to the second position.

In certain further aspects, the present disclosure relates to an automated microfluidic bioreactor for analyzing a sample to be reacted with one or more reagents. In certain aspects, a moveable magazine component comprises a plurality of microwells. Each microwell of the plurality of microwells comprises an inlet and a reservoir. At least one of the plurality of microwells is configured to hold one or more reagents used in an enzyme-linked immunosorbent assay (ELISA) to detect the one or more target biological analytes. The automated microfluidic bioreactor further comprises at least one microfluidic connector comprising at least one microfluidic channel having an aperture. The at least one microfluidic connector reversibly interfaces with at least one microwell of the plurality of microwells to establish fluid communication therebetween. A first motor is for translating the moveable magazine component with respect to the microfluidic connector. A second motor is included for translating the microfluidic connector from at least a first position where the microfluidic connector interfaces with or contacts the moveable magazine component to a second position where the microfluidic connector is not interfacing with/not in contact with the moveable magazine component. At least one of the translating the moveable magazine component or the translating of the at least one microfluidic connector is rotational, while the other translating is linear. At least one reactor chamber is in fluid communication with the microfluidic channel of the microfluidic connector. The at least one reactor chamber is configured to receive a test sample optionally comprising one or more target biological analytes. The automated microfluidic bioreactor further includes a pump or a pipette device for transporting fluids from the moveable magazine component through the microfluidic connector to the reactor chamber.

In one aspect, the at least one reactor chamber comprises a capture antibody capable of binding with at least one of the one or more target biological analytes and a detection antibody that comprises an indicator species.

In one further aspect, the one or more reagents includes a substrate, where a third microwell of the plurality of microwells comprises the substrate.

In one aspect, the microfluidic bioreactor further comprises at least one detector for analyzing fluids in the at least one reactor chamber.

In one aspect, the microfluidic bioreactor further comprises a wash system comprising a washing fluid and a second pump in fluid communication with the microfluidic connector.

In one further aspect, the wash system further comprises a fluid reservoir. A moveable magazine component may further comprise at least one wash inlet and at least one wash outlet for transporting the washing fluid from the fluid reservoir to the at least one microfluidic connector.

In one aspect, the outlet of each microwell of the plurality of microwells has a seal and the needle component is configured to puncture at least a portion of the seal.

In one aspect, at least a portion of the microwells of the plurality of microwells store the one or more reagents.

In one aspect, the microfluidic bioreactor further comprises one or more detectors for measuring one or more of luminescence, fluorescence, or colorimetry to determine an amount of the one or more target biological analytes present in the test sample.

In one aspect, the microfluidic bioreactor further comprises at least one processor configured to receive output from one or more detectors and configured to determine an amount of the one or more target biological analytes present in the test sample.

In one aspect, the microfluidic bioreactor further comprises at least one processor to control operation of at least one of: the first motor, the second motor, or the pump.

In one aspect, each microwell of the plurality of microwells has a respective volume of less than or equal to about 1 milliliter (mL).

In one aspect, a maximum dimension of the automated microfluidic reactor is less than or equal to about 25 cm and the automated microfluidic reactor is portable.

In one aspect, the at least one microfluidic connector is configured to engage at least one microwell of the plurality of microwells in the moveable magazine component. The at least one microfluidic channel comprises a plurality of microfluidic channels and the at least one reactor chamber comprises a plurality of reactor chambers each in fluid communication with a microfluidic channel of the plurality of microfluidic channels.

In certain other aspects, the present disclosure relates to an automated microfluidic bioreactor for analyzing a sample to be reacted with one or more reagents. The bioreactor comprises a moveable magazine component comprising a plurality of microwells. Each microwell comprises an inlet, a reservoir, and an outlet. At least one of the plurality of microwells is configured to receive a test sample optionally comprising one or more target biological analytes. At least one of the plurality of microwells is configured to hold one or more reagents used in an enzyme-linked immunosorbent assay (ELISA) to detect the one or more target biological analytes. The microfluidic bioreactor also includes at least one microfluidic connector comprising at least one microfluidic channel and a recess in which a microwell of the plurality of microwells reversibly seats. The at least one microfluidic connector comprises a needle component having at least one aperture to provide fluid communication between the recess and the microfluidic channel. The microfluidic bioreactor also has a first motor for translating the moveable magazine component with respect to the microfluidic connector, as well as a second motor for translating the microfluidic connector from at least a first position where the microfluidic connector contacts the moveable magazine component to a second position where the microfluidic connector is not in contact with the moveable magazine component. The microfluidic bioreactor also includes at least one reactor chamber in fluid communication with the microfluidic channel of the microfluidic connector. There is also a pump for transporting fluids from the moveable magazine component through the microfluidic connector to the reactor chamber.

In one aspect, the at least one reactor chamber comprises a capture antibody capable of binding with at least one of the one or more target biological analytes and a detection antibody that comprises an indicator species.

In one further aspect, the one or more reagents includes a substrate, where a third microwell of the plurality of microwells comprises the substrate.

In one aspect, the microfluidic bioreactor further comprises at least one detector for analyzing fluids in the at least one reactor chamber.

In one aspect, the microfluidic bioreactor further comprises a wash system comprising a washing fluid and a second pump in fluid communication with the microfluidic connector.

In one further aspect, the wash system further comprises a fluid reservoir. A moveable magazine component may further comprise at least one wash inlet and at least one wash outlet for transporting the washing fluid from the fluid reservoir to the at least one microfluidic connector.

In one aspect, the outlet of each microwell of the plurality of microwells has a seal and the needle component is configured to puncture at least a portion of the seal.

In one aspect, at least a portion of the microwells of the plurality of microwells store the one or more reagents.

In one aspect, the microfluidic bioreactor further comprises one or more detectors for measuring one or more of luminescence, fluorescence, or colorimetry to determine an amount of the one or more target biological analytes present in the test sample.

In one aspect, the microfluidic bioreactor further comprises at least one processor configured to receive output from one or more detectors and configured to determine an amount of the one or more target biological analytes present in the test sample.

In one aspect, the microfluidic bioreactor further comprises at least one processor to control operation of at least one of: the first motor, the second motor, or the pump.

In one aspect, each microwell of the plurality of microwells has a respective volume of less than or equal to about 1 milliliter (mL).

In one aspect, a maximum dimension of the automated microfluidic reactor is less than or equal to about 25 cm and the automated microfluidic reactor is portable.

In one aspect, the at least one microfluidic connector is configured to engage at least one microwell of the plurality of microwells in the moveable magazine component. The at least one microfluidic channel comprises a plurality of microfluidic channels and the at least one reactor chamber comprises a plurality of reactor chambers each in fluid communication with a microfluidic channel of the plurality of microfluidic channels.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 shows a schematic of an automated microfluidic reactor prepared in accordance with certain aspects of the present disclosure, where a moveable magazine component is disposed above a microfluidic connector.

FIGS. 2A-2C show one variation of a reservoir moveable magazine component and a microfluidic connector for an automated microfluidic reactor prepared in accordance with certain aspects of the present disclosure. FIG. 2A shows a schematic of a biosensor configuration including the reservoir moveable magazine component and the microfluidic connector from a perspective view with an inset showing a magnified portion.

FIGS. 2B-2C shows a top and side view of the reservoir moveable magazine component and microfluidic connector in FIG. 2A.

FIGS. 3A-3E show a reservoir moveable magazine component for an automated microfluidic reactor prepared in accordance with certain aspects of the present disclosure. FIGS. 3A-3C show top, side, and bottom views respectively of the design of the 3D printed reservoir moveable magazine component. FIGS. 3D-3E show the corresponding 3D printed versions of the reservoir moveable magazine component, as formed. FIG. 3D also includes an inset showing a washing/buffer fluid channel system.

FIGS. 4A-4B show a detailed sectional view (FIG. 4A) and a top view (FIG. 4B) of one microwell of a plurality of microwells shown in the reservoir moveable magazine component of FIGS. 3A-3C.

FIGS. 5A-5D show a 3D printing design of a microfluidic connector (green) along with the piercing needle component for an automated microfluidic reactor prepared in accordance with certain aspects of the present disclosure. FIG. 5A shows a perspective view with a magnified view of the needle component. FIG. 5B shows dimensions of the microfluidic connector. FIG. 5C shows a side sectional view of the microfluidic connector. The relative position between the needle component and the reservoir microwell during seal piercing is shown. FIG. 5D shows the corresponding 3D printed versions of the microfluidic connector, as formed.

FIG. 6 shows a protocol for conducting a sandwich-ELISA test, including preparation steps and detection steps.

FIG. 7 shows a view of the automated microfluidic reactor system according to certain aspects of the present disclosure.

FIGS. 8A-8C show a view of an entire automated microfluidic biosensor prepared in accordance with certain aspects of the present disclosure. FIG. 8A shows an overhead perspective view, FIG. 8B shows a side view, and FIG. 8C shows a capillary (reaction chamber) underneath the camera/detector for chemiluminescence reading.

FIGS. 9A-9D show results after reaction in an automated microfluidic reactor prepared in accordance with certain aspects of the present disclosure. FIG. 9A shows captured chemiluminescence images for 0, 200, 400, 700, and 1000 pg/mL concentrations of IL-6 (top panel, left to right) after reaction in an automated microfluidic reactor prepared in accordance with certain aspects of the present disclosure. FIG. 9B shows corresponding blue channel extracted version (bottom panel). FIG. 9C shows intensity readings of the capillaries for the concentrations mentioned in FIG. 9A. FIG. 9D show a calibration curve for the concentrations with an R² value of 0.96. The intensity is taken at the center position of each capillary.

FIG. 10 shows a schematic of an automated microfluidic reactor prepared in accordance with certain other aspects of the present disclosure, where a moveable magazine component is disposed below a multi-channel microfluidic connector providing multiplex analysis capability.

FIG. 11 shows a top view of a multi-channel microfluidic connector in fluid communication with a plurality of reaction chambers in fluid communication with a multi-channel pipette device.

FIG. 12A shows a side view of one variation of a microfluidic connector comprising at least one microfluidic channel having an aperture in fluid communication with a microwell including a reservoir region containing a reagent according to certain aspects of the present disclosure.

FIG. 12B shows a side view of another variation of a microfluidic connector comprising at least one microfluidic channel having an aperture that defines a needle in fluid communication with a microwell including a reservoir region containing a reagent according to certain aspects of the present disclosure.

FIG. 13 shows a top view of a reservoir moveable magazine component for an automated microfluidic reactor prepared in accordance with certain aspects of the present disclosure, where the moveable magazine component includes an array of microwells that contain distinct reagents in distinct rows or lines interacting with a multichannel microfluidic connector connected to a multichannel reaction chamber.

FIG. 14 shows a top view of show a reservoir moveable magazine component for an automated microfluidic reactor prepared in accordance with certain aspects of the present disclosure, where the moveable magazine component includes an array of microwells that contain distinct rows or lines having alternating distinct reagents and interspersed microwells serving as disposal microwells having a wicking material disposed therein for interacting with a multichannel microfluidic connector connected to a multichannel reaction chamber.

FIG. 15 shows a side view of a variation of a microfluidic connector comprising at least one microfluidic channel having an aperture for fluid communication with a microwell containing a wicking mateiral in a reservoir region and serving as a disposal well according to certain aspects of the present disclosure.

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

In this application, the term “processor,” “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. Any functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

None of the elements recited in the claims are intended to be a means-plus-function element within the meaning of 35 U.S.C. § 112 (f) unless an element is expressly recited using the phrase “means for,” or in the case of a method claim using the phrases “operation for” or “step for.”

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges.

Example embodiments will now be described more fully with reference to the accompanying drawings.

In various aspects, the present disclosure contemplates an automated microfluidic reactor for analyzing a sample by a reaction with one or more reagents. The sample may contain one or more target analytes, such as a molecule, compound, or biological species of interest. Thus, the reaction with one or more reagents may be used to determine a presence of such target analytes and/or to quantify an amount (e.g., determine a concentration) of such target analyte(s) within the sample. As will be described further below, the automated microfluidic reactor may be an inorganic or organic reactor. While the automated microfluidic reactor is not limited to biosensors or bioreactors, which may conduct biological assays (bioassays), in certain variations, the automated microfluidic reactor is a bioreactor and/or biosensor. Bioassays, such as enzyme-labeled or fluorescence-labeled immunoassays, are used to detect and quantify specific soluble substances in a biological sample. For example, enzyme-linked immunosorbent assay (ELISA) and its variations (such as fluorescence-label immunoassay) are commonly used biological assays (bioassays) that can quantify target species (such as biomolecules) in a sample.

In traditional sandwich-type ELISA, capture antibodies are first immobilized on the surface of solid materials (such as glass, metal, or plastics/polymers). For example, for a conventional sandwich-type ELISA assay, one or more capture antibodies may be immobilized onto surfaces in one or more wells of a 96-well plate. A sample, which potentially contains the analyte of interest, is introduced in a manual or automated process (e.g., by pipetting) into a well having a surface coated with the capture antibody, where a complex between the capture antibody and analyte within the sample is formed typically during an incubation step. After the binding of the target molecules (such as cytokines, interleukin (IL)-6, IL-10, tumor necrosis factor (TNF)), the well may be washed using a wash buffer to remove unbound analyte. Next, detection antibodies that are conjugated with enzymes (such as horseradish peroxidase (HRP)), are added to bind with the target molecules bound to the capture antibodies. Finally, substrates are added to interact with enzymes. The enzyme-substrate reaction makes the substrate emit light (such as chemiluminescence or chemifluorescence) or change its color (colorimetric detection). Alternatively, the detection antibodies are conjugated with fluorescent reporters, such as dyes and quantum dots, or with plasmonic nanoparticles (i.e., metal nanoparticles), where the emitted light from dyes or quantum dots, or the scattered light from plasmonic nanoparticles are detected to quantify the target analyte. Such detection is called analog detection, since the emitted or scattered light intensity, or the transmitted light (in colorimetric detection) is used to quantify the target analyte.

In certain aspects, the present disclosure contemplates an automated, ELISA-based biosensor that is cost-effective and can be used at point of care. The device has relatively small dimensions, for example, having a maximum dimension of less than or equal to about 25 cm, making it compact and portable. The device is inexpensive, for example, costing at least an order of magnitude less than conventional devices to manufacture, which can be further reduced by industrial and mechanical optimizations for mass production purposes. In certain aspects, one or more components may be formed from an additive manufacturing process, such as three-dimensional (3D) printing. The 3D printed components or parts may be made from a 3D printing technique, such as stereolithography (SLA) by using a resin (e.g., a clear resin). The 3D printed part may be disposable and inexpensive, as will be described further herein. The 3D printed parts may be formed of one or more polymeric materials.

In various aspects, an automated microfluidic reactor is contemplated for analyzing a sample to be reacted with one or more reagents. The reactor may comprise a moveable magazine component comprising a plurality of microwells. Each microwell of the plurality of microwells comprises an inlet or opening and a reservoir. At least one of the plurality of microwells is configured to hold one or more reagents. Further, at least one microfluidic connector is provided that comprises at least one microfluidic channel having an aperture. The at least one microfluidic connector reversibly interfaces with at least one microwell of the plurality of microwells to establish fluid communication therebetween. The automated microfluidic reactor further includes a first motor for translating the moveable magazine component with respect to the microfluidic connector, as well as a second motor for translating the microfluidic connector from at least a first position where the microfluidic connector contacts the moveable magazine component to a second position where the microfluidic connector is not in contact with the moveable magazine component. The translating may include rotation, moving linearly, or side-by-side, or the like. In certain variations, at least one of the translating the moveable magazine component or the translating of the at least one microfluidic connector is rotational and the other is linear. In certain variations, the translating of the moveable magazine component occurs by rotating via operation of the first motor, while the translating of the at least one microfluidic connector occurs by linear translating via operation of the second motor.

The automated microfluidic reactor further comprises at least one reactor chamber in fluid communication with the microfluidic channel of the microfluidic connector. The at least one reactor chamber is configured to recive a test sample that optionally contains one or more analytes. The automated microfluidic reactor may also include either a pump or other means for transporting fluids from the moveable magazine component through the microfluidic connector to the reactor chamber (e.g., a pipette or other device). In certain variations, the moveable magazine component may be disposed over the at least one microfluidic connector, while in other variations, the at least one microfluidic connector may be disposed over the moveable magazine component.

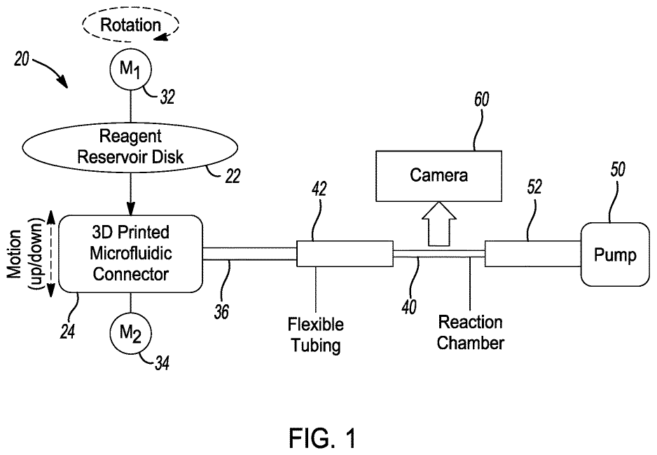

An automated microfluidic reactor 20 for analyzing a sample to be reacted with one or more reagents according to certain aspects of the present disclosure is shown in FIG. 1, where the moveable magazine component is disposed over the at least one microfluidic connector and the system includes a pump, as further described herein. The reactor 20 includes a reservoir moveable magazine component 22 configured to be rotated. The reactor 20 also includes at least one microfluidic connector 24 in fluid communication with the reservoir moveable magazine component 22. In this variation, the at least one microfluidic connector 24 is configured to translate linearly, for example, to move up and down. A first motor 32 (designated M₁) is mechanically connected to the reservoir moveable magazine component 22 and serves to rotate the reservoir moveable magazine component 22 with respect to the microfluidic connector 24. A second motor 34 (designated M₂) is mechanically connected to the microfluidic connector 24 and serves to linearly translate the microfluidic connector 24 from at least a first position where the microfluidic connector 24 contacts the reservoir moveable magazine component 22 to a second position where the microfluidic connector 24 is not in contact with the reservoir moveable magazine component 22. In other variations, the reservoir moveable magazine component 22 may move linearly, or side-by-side, rather than rotationally, so that the reservoir moveable magazine component 22 moves horizontally using a step motor based linear stage, positied above the microfluidic connector 24 in a sequential manner. The microfluidic connector 24 may be rotated by a motor in such a configuration.

The microfluidic connector 24 includes a microfluidic channel 36 that may be connected to at least one reaction or reactor chamber 40 via a first connector 42, which may be a microfluidic chamber to establish fluid communication between the microfluidic channel 36 of the microfluidic connector 24 and the reactor chamber 40. A cross-sectional shape of the reactor chamber 40 may be round or non-round, including a rectangular shape. The reactor chamber 40 may be formed from a segment of flexible tubing, a glass capillary, and the like, by way of example. In alternative variations, instead of using tubing or capillaries with a circular or round cross section, reaction chambers may be formed as microfluidic channels (or chambers) fabricated on a planar surface (e.g., defining channels having a round or circular cross-sectional shape on a microfluidic chip), which is more suitable for mass production. Further, additional reaction chambers/channels can be added to accommodate multiplexed detection.

The reactor 20 further includes a pump 50 for transporting fluids within the system, for example, moving fluids from the reservoir moveable magazine component 22 through the microfluidic connector 24 to the reactor chamber 40. The pump 50 may further faciliate other fluid flow, including evacuating the reactor chamber 40 among other regions of the reactor 20 system. Pump 50 is connected to the reactor chamber 40 via a second connector 52, which may be a segment of flexible tubing and establishes fluid communication between the various regions of the reactor 20 system, including between the reactor chamber 40, the microfluidic channel 36, the microfluidic connector 24, and the reservoir moveable magazine component 22, by way of example. The reactor 20 may also have at least one detector 60, for example, a camera, for analyzing fluids in the reactor chamber 40.

As noted above and will be described further herein, the automated microfluidic reactor 20 in FIG. 1 may be used as a bioreactor or biosensor, where the reservoir moveable magazine component 22 may have a plurality of microwells or reservoirs to contain one or more reagents (not shown in FIG. 1). Further, at least one microwell may receive a test sample for processing in the reactor. Thus, the reactor 20 can include the reservoir moveable magazine component 22 and the microfluidic connector 24, which may be connected to at least one selected individual microwell or reservoir in the reservoir moveable magazine component 22. However, the present technology is not limited to only one microfluidic connector 24 being engaged and connected with one microwell or reservoir. In certain variations, multiplexed, multiple microfluidic connectors 24, may be connected to multiple select microwells. In alternative variations, one microfluidic connector 24 may be connected to multiple microwells, and the like. Further, microfluidic connector(s) 24 are connected to a reactor chamber 40 that may be an ELISA-based reaction chamber. The microfluidic connector 24 can move up and down vertically to connect and disconnect the microwell(s) in the reservoir moveable magazine component 22 to the reactor chamber 40. Hence, this microfluidic connector 24 acts like a microfluidic valve, except that its operation is through its mechanical movement. Through the microfluidic connector 24, the sample/reagent fluids are pulled from the microwell/reagent reservoir in the reservoir moveable magazine component 22 using the pump 50. Upon arrival of the sample/reagent fluid into the reaction chamber 40, the pump 50 stops, and the sample/reagent liquid may remain within the reaction chamber 40, for example, for incubation. The pump 50 may be a syringe pump, which can pull waste liquids into a disposable syringe.

A signal may be taken by reading the chemiluminescence intensity on the transparent reaction/reactor chamber 40 with the at least one detector 60, which may be eventually translated into analyte concentration. As will be appreciated, the detector 60 may be configured to measure one or more of luminscence, fluorescence, or colorimetry, or other detectable levels of emission to determine an amount of the one or more target analytes present in the test sample.

In various aspects, while not shown, the automated microfluidic reactor 20 device may be part of a system that further includes one or more processors in electrical communication with one or more components, including the detector(s) 60, motor(s), pump(s), and the like. For example, the processor may be a computer processing unit (CPU) associated with the system that includes the automated microfluidic reactor 20 that may be operated with various modules, as appreciated by those of skill in the art. In other variations, the processor may be integrated into the automated microfluidic reactor 20 (e.g., a microprocessor). In certain aspects, the processor is configured to receive and record a plurality of measurements for example, of one or more of luminscence, fluorescence, or colorimetry, taken by the detector(s) 60 and may include discrete or continuous measurements. In certain aspects, the processor is optionally further configured to calculate a concentration of one or more target analytes present in the test sample. In certain aspects, this analysis may be conducted in the processor by using an image analysis software program, such as Image J™ software. For example, chemiluminescence images of the reactor chamber can be recorded and then analyzed by such software. Thus, the reactor system may be an automated system.

Once the incubation time is over and detection and measurements have been taken, the pump 50 continues pulling the sample/reagent to exit the reaction chamber 40 into a waste stream (not shown).

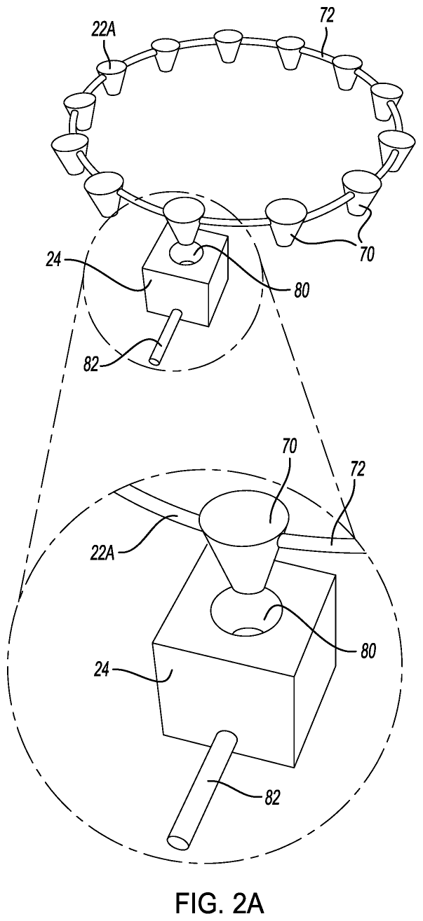

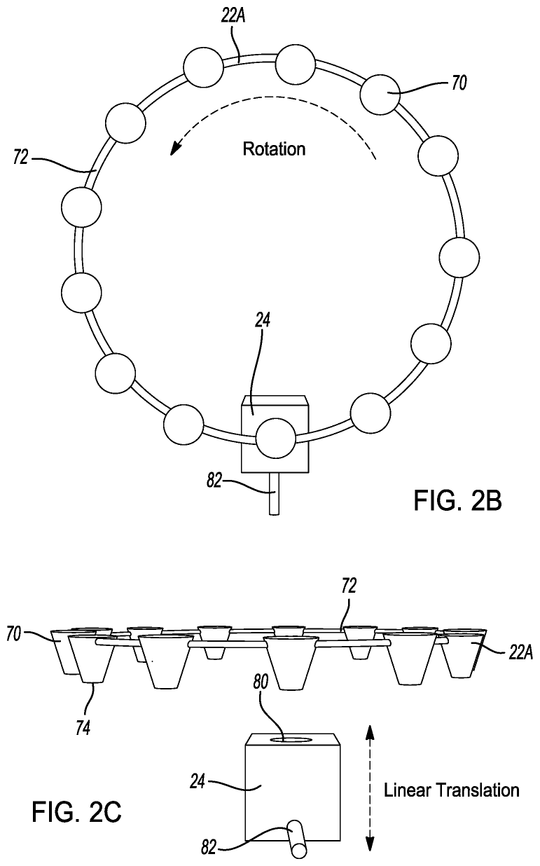

FIGS. 2A-2C show a first variation of a moveable magazine component 22A, shown as a reservoir disk component, and the microfluidic connector 24 like in FIG. 1 interfacing with one another, where the moveable magazine component 22A is above the microfluidic connector 24. As will be described further below, at least one motor translates the moveable magazine component 22A with respect to the microfluidic connector, which may include rotation (e.g., rotating the reservoir disk component), or moving linearly, or side-by-side, or the like. Further, a second motor translates the microfluidic connector 24 from at least a first position where the microfluidic connector 24 contacts the moveable magazine component 22A (e.g., reservoir disk component) to a second position where the microfluidic connector 24 is not in contact with the moveable magazine component. In the variation shown, the reservoir moveable magazine component 22A has a plurality of microwells 70 connected by a ring connector 72 that spans between each respective microwell 70. As used herein, “microscale” refers to a structure having at least one dimension that is less than about 500 micrometers (μm), optionally less than about 400 micrometers, optionally less than about 300 micrometers, optionally less than about 200 micrometers, optionally less than about 150 micrometers, optionally less than about 100 micrometers, and in certain variations, optionally less than about 50 micrometers. As used herein, reference to a microscale, microwell, microreservoir, a microchannel, a microfluidic channel, or a microstructure, encompasses smaller structures, such as those being on a nanoscale.

A volume (e.g., storage volume) of the microwell 70 may be up to hundreds of milliliters, for example, less than or equal to about 1 liter, optionally less than or equal to about 750 milliliters, optionally less than or equal to about 500 milliliters, optionally less than or equal to about 250 milliliters, optionally less than or equal to about 100 milliliters, optionally less than or equal to about 50 milliliters, optionally less than or equal to about 25 milliliters, optionally less than or equal to about 1 millilite. In certain variations, the storage volume of the microwell 70 may optionally be less than or equal to about 750 microliters, optionally less than or equal to about 500 microliters, optionally less than or equal to about 250 microliters, optionally less than or equal to about 100 microliters, optionally less than or equal to about 50 microliters, optionally less than or equal to about 25 microliters, and in certain aspects, optionally less than or equal to about 1 microliter. In certain aspects, a storage volume may be greater than or equal to about 1 microliter to less than or equal to about 1 liter.

A microwell, microreservoir, or microfluidic channel is formed in or on a substrate that has a cross-sectional area and volume sufficient to permit receiving, storing, and/or transferring materials, including fluids. Fluids include liquids, gases, vapors, and the like. A microwell may define a discrete shape with a volume distinct from other microwells. Thus, a microwell may have dimensions such that the length of the structure forms the greatest dimension, for example, a groove (an open shape) or channel (a structurally closed geometry). The microwell may have a variety of shapes. In certain aspects, one or more microwells may define a symmetric shape, such as a conical, frustoconical, rectangular, cylindrical shape. Each microwell of the plurality of microwells comprises a reservoir to receive or store fluid and an inlet or opening through which the reservoir may be accessed. In certain variations, the microfluidic channels may be fully enclosed structures defining a void region that permits fluid communication there through, as described further herein. The microfluidic channels may have a variety of cross-sectional shapes, including circular, round, or oval (forming a tube or cylindrical shape), rectangular, and the like.

As noted above, the reservoir moveable magazine component 22A is a structure capable of being rotated about a central axis, but in this variation, merely includes a ring structure defined by the ring connector 72 having a plurality of microwells 70. In other variations, the reservoir moveable magazine component may be a plate or disk component in which a plurality of microwells is defined. The plurality of microwells 70 may be evenly distributed around its circumference, in certain variations. It should be noted that while the microwells 70 are shown to have a conical or frustoconical shape, for example, thus having a shape capable of receiving a conventional tip of a pipette or other injection device from one side, other shapes may also be used as a reservoir to hold fluids/liquids. Reservoir moveable magazine components prepared in accordance with various aspects of the present disclosure may be formed of a polymer and manufactured by various processes, including 3D printing, such as stereolithography (SLA) printing, or injection molding. For example, one suitable highly-durable 3D printing resin that is contemplated is Tough 2000™ resin, which can be used for more durable printing of much thinner disks for reduced material cost and higher printing speed. Another suitable polymer is Clear Resin V4.1.

In certain variations, the microfluidic connector 24 may include a recess 80. For variations where the reservoir moveable magazine component (e.g., 22A) is disposed above the microfluidic connector (e.g., 24), the recess 80 in the microfluidic connector 24 may have a complementary shape to the shape of the microwells 70, so that a respective microwell 70 may reversibly seat within the recess 80 in the variation shown in FIG. 2A. In certain variations, the microfluidic connector 24 may translate linearly with respect to the reservoir moveable magazine component 22A. In other variations, the microfluidic connector 24 may move rotationally with respect to the moveable magazine component 22A, so that the moveable magazine component 22A instead translates linearly, for example, moving up and down.

The microfluidic connector 24 further comprises at least one microfluidic channel 82 that is in fluid communication with the recess 80 and through which fluid can pass. The microfluidic channel 82 includes at least one opening or aperture 84 through which fluid may move. The at least one microfluidic connector 24 reversibly interfaces with respective microwell 70 to establish fluid communication therebetween. It should be noted that in alternative variations not shown, it is contemplated that the reactor chamber 40 may be directly integrated into the microfluidic connector 24, for example, substituting for the microfluidic channel 82.

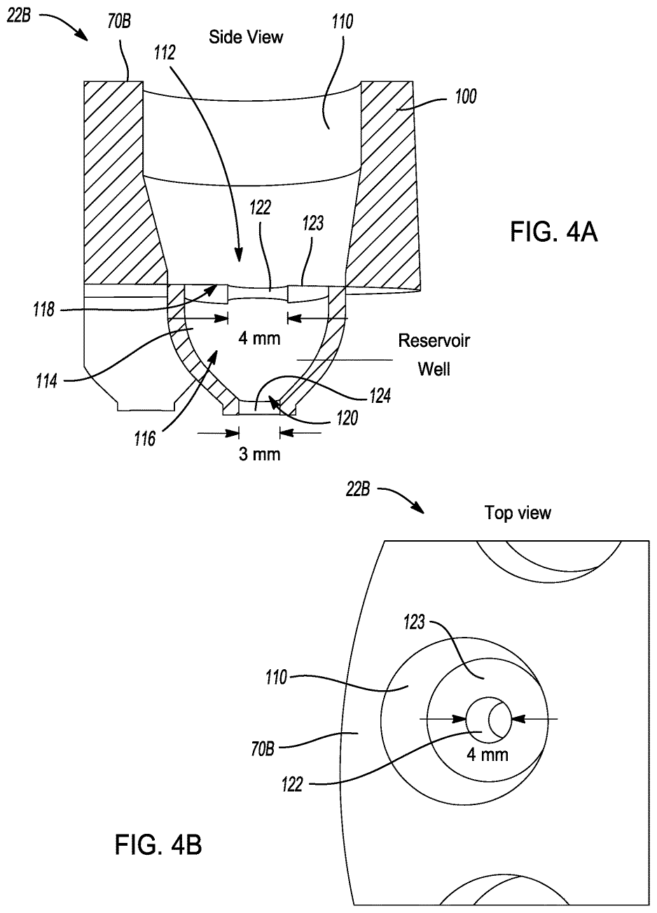

As will be described further in the context of FIGS. 5A-5D, the microfluidic connector 24 may further include at least one needle component 90 that itself defines at least one needle aperture 92 to establish a pathway for fluids to travel from the recess 80 through the aperture 84 into the microfluidic channel 82. The at least one needle aperture 92 may be a single aperture or multiple needle apertures 92, for example, four needle apertures as shown. The needle component 90 may further include a needle tip 94, which as described further below, may be configured to puncture a seal disposed over a terminal or lower opening 74 of the corresponding microwell 70. FIG. 5C shows dimensions of the microfluidic connector 24 are 18 mm by 18 mm, by way of example, while a recess 80 has a diameter of about 11.6 mm.

As best seen in FIG. 5C, a microwell 70 may contain reagent (e.g., fluid/liquid) stored in a reservoir and that may be seated within the recess 80 of the microfluidic connector 24. As will be appreciated, all of the dimensions described herein, including of the microfluidic channels and microwells may be further reduced to minimize reagent consumption. Microfluidic connectors 24 prepared in accordance with various aspects of the present disclosure may be formed of a polymer and manufactured by various processes, including 3D printing, such as stereolithography (SLA) printing, or injection molding. For example, one suitable highly-durable 3D printing resin that is contemplated is Tough 2000™ resin, which can be used for more durable printing of much thinner parts for reduced material cost and higher printing speed. Another suitable polymer is Clear Resin V4.1. It will be appreciated that while one microfluidic connector 24 with one microfluidic channel 82 and one needle component 90 is shown, for multiplexed detection, one microfluidic connector 24 with one needle component 90 may be connected to one larger reservoir (e.g., microwell 70) and once the liquid is pulled, it can be distributed through branching channels into multiple reaction chambers, each associated with one target analyte.

Thus, FIGS. 2A-2C show the detailed configuration of the reservoir moveable magazine component 22A having microwells 70 (or reagent reservoirs) and microfluidic connector 24, where the microwells 70 are made on a disk. In certain variations, each may be designated for storing a specific reagent used in the ELISA protocol or used for adding a sample under test. Through the rotation of the reservoir moveable magazine component 22A, the desired microwell 70 is placed on top of the microfluidic connector 24. FIGS. 2B-2C show the top and the side view of the biosensor setup, respectively. The vertical (up and down) movement of the microfluidic connector 24 and the rotational movement of the reservoir moveable magazine component 22A are the only two mechanical movements for the operation of the biosensor, which can be implemented easily and cost-effectively using a linear actuator and a step motor, respectively.

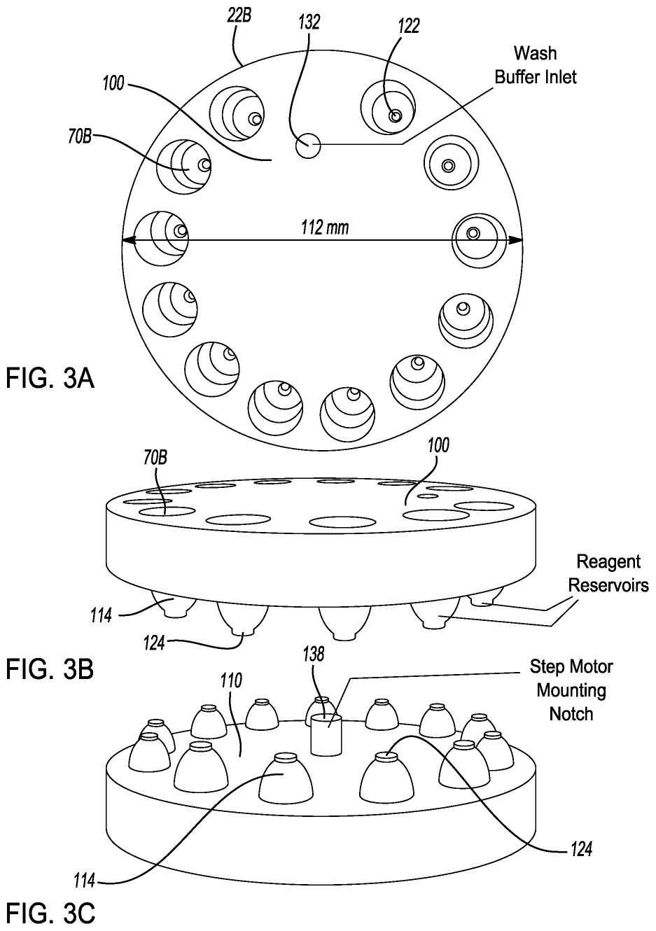

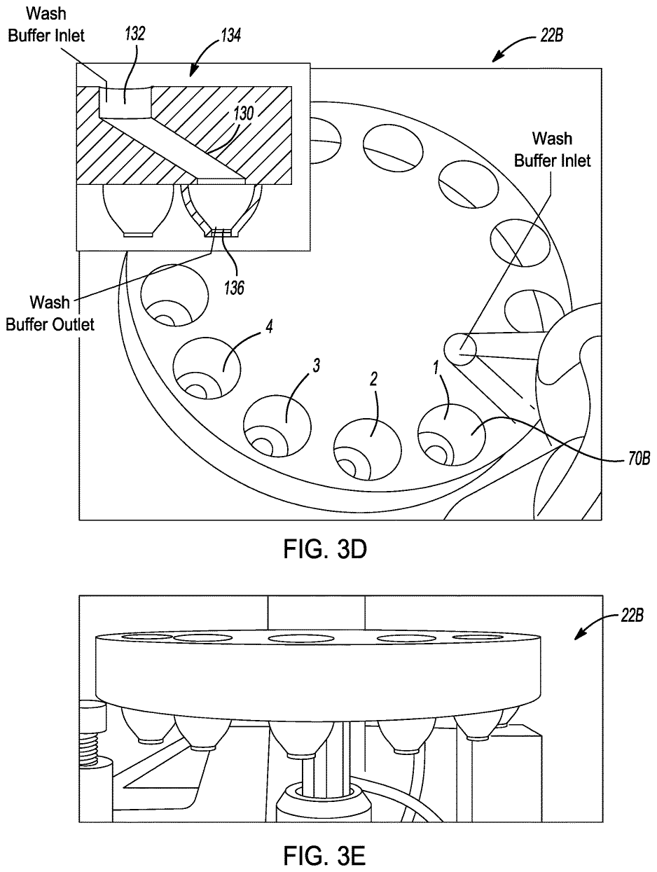

FIGS. 3A-3E and 4A-4B show another variation of a reservoir moveable magazine component 22B that may be used with the previously described microfluidic connector 24 like in FIGS. 1 and 5A-5D. To the extent that the features of the reservoir moveable magazine component 22A in FIGS. 2A-2C are the same as those of reservoir moveable magazine component 22B, the same reference numbers are used herein and for brevity will not be described again in detail unless otherwise noted. The reservoir moveable magazine component 22B has a plurality of microwells 70B formed in a disk-shaped round solid body portion 100 at predetermined intervals (evenly spaced apart along a circumference of the reservoir moveable magazine component 22B). FIGS. 4A-4B show a detailed view of one select microwell 70B, which defines a first region 110 having two open ends that form a hollow or open channel formed in the round solid body portion 100. In certain aspects, material in this first region 110 may be removed (e.g., carved out) to remove material processing time and expense, for example, to save 3D-printing time and material. Further, the carved area in the first region 110 provides easy access to a second region 114 when using a pipette. It is contemplated there should more robust and tougher resins be used to form the reservoir moveable magazine component 22B, it may be thinner and the first region 110 may be eliminated from the design.

A lower or terminal end 112 of the first region 110 transitions into a second region 114 that is configured to hold and/or store a fluid in an internal open storage volume 116, thus serving as a reservoir well. In certain aspects, the open storage volume may be any of those described above, for example, greater than or equal to about 1 microliter (μL) to less than or equal to about 1 liter (L). As shown, the storage volume is about 364 microliters (μL). The second region 114 defines a first side 118 (e.g., upper side) and a second side 120 (e.g., a lower side). The terminal end 112/first side 118 may have a first opening 122 through which one or more fluids/liquids can be introduced, including a sample to be tested. In certain variations, the first opening 122 may be closed or sealed, for example, having a removable plug or fluid-tight seal (e.g., foil or polymeric film) disposed therein (not shown). In certain variations, where a seal is used over the first opening 122 on the first side 118, it may be punctured or otherwise opened externally or alternatively, punctured by the needle tip 94 of the needle component 90 of the microfluidic connector 24, which may be designed to have a dimension making it capable of extending up to and/or past the first side 118, when the microwell 70B is seated in the microfluidic connector 24. In other aspects, the plug or seal may be removed manually by an operator of the device. As shown in FIGS. 4A-4B, the first opening 122 may be surrounded by a concentric flange 123 attached to the wall and defines a diameter of about 4 mm, by way of non-limiting example. In certain other variations, the first opening 122 may omit a flange or have a smaller flange/lip 123 and can be as wide as the external wall of the first region 110 transitioning to the second region 114.

The second region 114 further includes a second opening 124 on the second side 120 (e.g., a lower side). Again, the second opening 124 permits one or more fluids/liquids to flow and pass therethrough from the internal storage volume 116. The second opening 124 may be closed or sealed, for example, having a removable plug or seal disposed thereon (not shown). In certain variations, where a seal is used to close the second opening 124 on the second side 120, it may be punctured or otherwise opened by the needle tip 94 of the needle component 90 designed to extend past the second side 120 when the microwell 70B is seated in the microfluidic connector 24. As shown in FIGS. 5A-5B, the second opening 124 may have a diameter of about 3 mm, by way of non-limiting example.

With renewed reference to FIGS. 3A-3E, the body portion 100 of the reservoir moveable magazine component 22B may have a diameter of about 112 mm, by way of non-limiting example. Further, as shown in FIG. 3A and the inset of FIG. 3D, an optional wash fluid/buffer channel 130 may be formed in the body portion 100. The wash fluid/buffer channel 130 may have an inlet 132 on an upper surface 134 of the reservoir moveable magazine component 22B and the channel 130 is in fluid communication with a modified microwell having the same lower shape as the other microwells 70B so that they may reversibly seat within the recess 80 as the microfluidic connector 24, but being dedicated to the wash fluid/buffer system and having an outlet 136 through which the wash fluid/buffer fluid can pass. As will be described further below, a source of wash fluid/buffer solution may be introduced into the inlet 132 so that it flows through the channel 130 and through outlet 136 into the reservoir moveable magazine component 22B and downstream into the remainder of the system (e.g., into reaction chamber 40).

Thus, in this variation, the reservoir moveable magazine component 22B has a special design for wash buffer inlet 132 to receive wash buffer from a flexible tubing placed right above it. The flexible tubing is connected to a large wash buffer reservoir (such as a 50 ml conical centrifuge tube, described further below). The outlet 136 of the wash buffer is on the same circumference as other outlets for reagent reservoir microwells. The inlet and outlet for wash buffer are not sealed. By incorporating a separate wash buffer reservoir and the associated inlet/outlet, ample wash buffer fluid is provided for thorough rinsing, as there are often dead volumes in microfluidics (such as in the microfluidic connector 24 and ELISA reaction chamber 40) that have residual samples and reagents that affect ELISA measurements. The reservoir moveable magazine component 22B design and the buffer arrangement are highly flexible. The number of microwells on the reservoir moveable magazine component 22B can be increased or decreased, depending on the needs. Additionally, while not shown, as will be appreciated, if multiple types of buffers are needed, multiple buffer sources (e.g., several large tubes) can be connected to corresponding inlets and outlets on the reservoir moveable magazine component 22B.

With reference to FIG. 3C, a lower side of the body portion 100 of the reservoir moveable magazine component 22B has a centrally disposed mounting feature 140, such a notch, so that the reservoir moveable magazine component 22B can be mounted onto the step motor and be rotatingly engaged therewith.

With regard to FIG. 3D, a plurality of microwells 70B are respectively labeled “1,” “2,” “3,” and “4.” Each of these microwells may contain/store a different reagent used in sequence for the downstream reactions, as will be further described herein. Further, one of the microwells, whether containing a reagent fluid/liquid or not, may receive a sample. Thus, FIGS. 3A-3C show a design of one variation of reservoir moveable magazine component 22B, as well as FIGS. 3D-3E that show its 3D printed version. In this variation, by way of non-limiting example, the reservoir moveable magazine component 22B contains 12 microwells 70B (each having a top first opening 122 diameter of about 4 mm and a bottom exit second opening 124 diameter of about 3 mm, as described in the context of FIGS. 4A-4B) that can store reagents and receive samples. However, smaller volumes of microwells can also be formed and/or accommodated.

As noted above, because the bottom second opening 124 of the microwell 70B (e.g., reservoir well) is sealed, in order to release the reagent in the reservoir, the seal at the bottom/second opening 124 of the microwell 70B can be removed or pierced by the needle tip 94 of the needle component 90 disposed in the middle of the microfluidic connector 24, which can be 3D printed together with other parts in the microfluidic connector 24. Upon piercing through the seal of the reagent reservoir, the reagent fluid is pulled from the reagent reservoir (microwell 70B) using the pump 50 in FIG. 1 through the holes or needle apertures 92 on the side of the needle component 90. In many other biosensing device designs, piercing a seal is accomplished with a permanent needle, which requires thorough rinsing between each step, thus involving many mechanical movements of the needles and dedicated mechanical components (such as a robotic arm to move the needle up and down to pierce into and pull out of the reagent reservoir, and to move the needle laterally for rinsing). For the present design, no dedicated mechanical components are needed. The needle can be thoroughly rinsed by the wash buffer concomitantly with the rinsing of other microfluidic parts (such as microfluidic connector 24 and ELISA reaction chamber 40, etc.).

The reagents can be stored and shipped within microwells (e.g., 70, 70B) of the disposable reservoir moveable magazine component 22B, eliminating the need for laboratory preparation and trained and expert operation. In one variation, the ELISA device has dimensions of approximately 19 cm by 24 cm, with a highest part of 14 cm. The bioreactor device is used to detect various concentrations of the Interleukin-6 (IL-6) analyte as a model system, with an R² value of 0.96. In this manner, the present disclosure provides a highly cost-effective, point-of-care, automated, and sensitive ELISA-based biosensors, which is compact and transportable and thus, can be used for field-deployable applications.

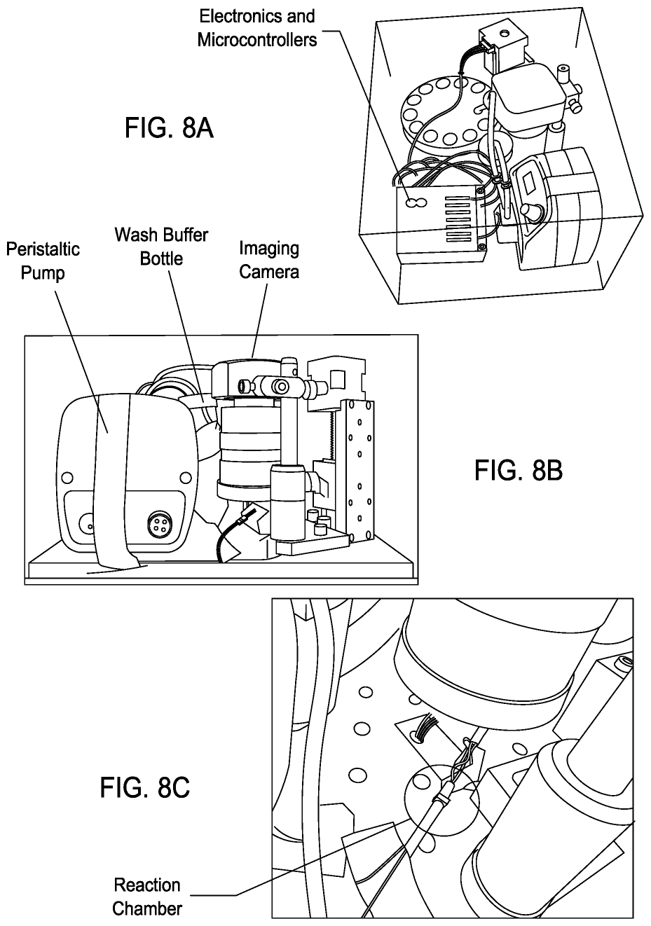

FIG. 10 shows another variation of automated microfluidic reactor 20C for analyzing a sample to be reacted with one or more reagents according to certain aspects of the present disclosure, where a moveable magazine component 22C, shown as a reservoir disk component, is disposed below at least one microfluidic connector 24C. To the extent that the features of the various components of the automated microfluidic reactor 20C are the same as those of earlier variations of the automated microfluidic reactor (e.g., 20), the same reference numbers are used herein and for brevity will not be described again in detail unless distinctions in the design are otherwise noted. The reservoir moveable magazine component 22C is configured to translate, for example, to be rotated by a first motor (not shown). The moveable magazine component 22C may include a plurality of microwells 70C, which may be defined on a disk or plate 151 and may together define a pattern or array of microwells 70C. As shown, the microwells 70C define a plurality of discrete and spaced apart rows or lines extending radially from a center of the round plate/disk to a circumferential perimeter. However, as will be appreciated, other patterns of microwells 70C are likewise contemplated, including by way of example, concentric rings of microwells 70C (not shown). Each microwell 70C includes an inlet and a reservoir defining a storage region, as will be described further below. At least one of the plurality of microwells 70C in the moveable magazine component 22C is configured to hold one or more reagents.

The at least one microfluidic connector 24C can reversibly be in fluid communication with the reservoir moveable magazine component 22C and more particularly with the reservoir region through the inlet of each microwell 70C. The at least one microfluidic connector 24C may translate from a first position where it is not interfacing with the moveable magazine component 22C to a second position where it is interfacing with at least one microwell 70C on the moveable magazine component 22C. In this variation, the at least one microfluidic connector 24C is configured to translate linearly, for example, to move up and down, for example, by the second motor (not shown). The moveable magazine component 22C may likewise translate when the at least one microfluidic connector 24C is in the first position. The moveable magazine component 22C may rotate when the at least one microfluidic connector 24C is disengaged in the first position.

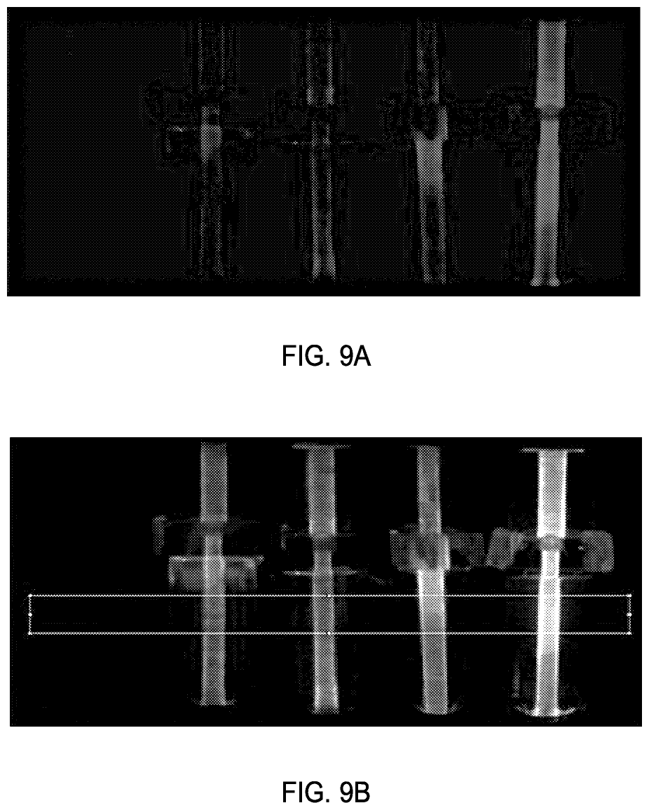

Further, the reactor 20C enables a multiplexed configuration, including a plurality of microfluidic connectors 24C disposed on a multi-channel component 150. Each microfluidic connector 24C may include a reactor chamber 40C that may be a separate component (not shown) or may be directly integrated into each microfluidic connector 24C (for example, as shown in FIG. 11 substituting for the microfluidic channel 82 shown in previous embodiments). Each microfluidic connector 24C may have at least one opening or aperture 152 on a first end for transferring or moving fluid within or through each, for example, in the microfluidic connector/reactor chamber 40C. Each microfluidic connector 24C may further have at least one second opening 154 on a second end, also for transferring or moving fluid within or through each microfluidic connector 24C, for example, in the microfluidic connector/reactor chamber 40C. The reactor chamber 40C region of the microfluidic connector 24C may be transparent to light in a range of wavelengths of interest like the previous embodiments. The automated microfluidic reactor 20C includes a detector in the form of an optical reader 155, for detection of the contents in the plurality of reactor chambers 40C in the microfluidic connector 24C, as described above. A data processing system 156 is associated with the optical reader 155 for processing its output, as described previously above.

The automated microfluidic reactor 20C may be associated with different fluid/liquid handling systems. In one variation, the liquid handling mechanism may be a pump, such as a syringe pump, versus a pipette. The multi-channel component 150 is shown in FIG. 11 attached to a multi-channel pipette device 160. More specifically, the multi-channel pipette device 160 includes a plunger mechanism 162 in fluid communication with a manifold 164 attached to a plurality of pipette tips 166. The plurality of pipette tips 166 may be connected to/in fluid communication with a plurality of conduits or connectors, such as flexible tubes 168. The flexible tubes 168 are respectively in fluid communication with the second end 154 of the microfluidic connectors 24C in the multi-channel component 150. In this manner, the multi-channel pipette device 160 can draw or transfer fluids from the microwells 70C in the moveable magazine component 22C up into the microfluidic connectors 24C/reactor chambers 40C via fluid communication through the plurality of flexible tubes 168.

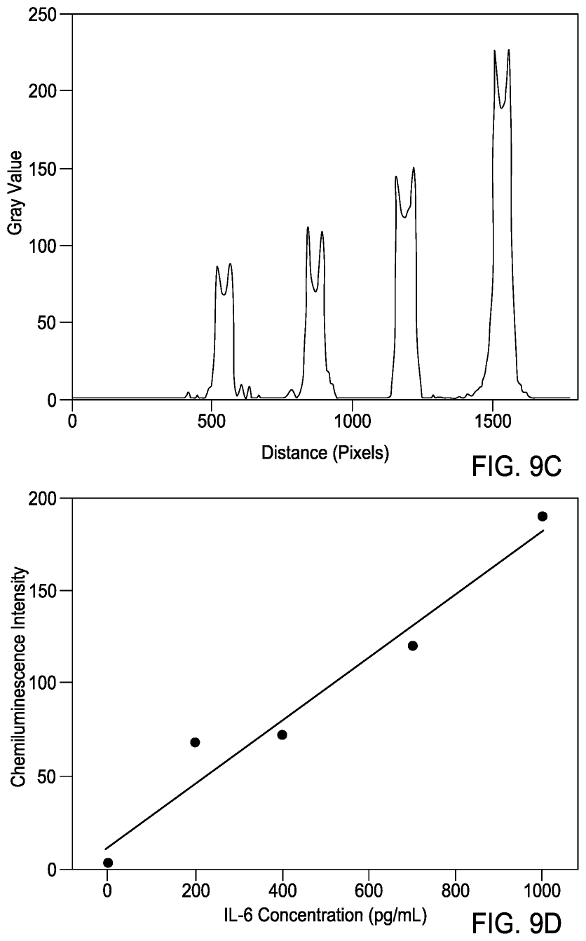

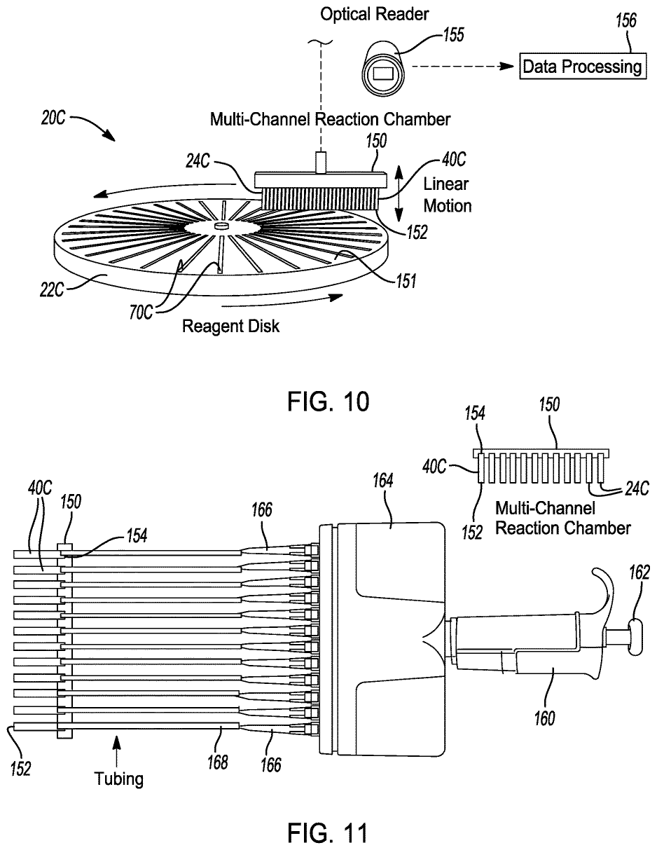

With reference to FIG. 12A, a first embodiment of a single microwell 70C is shown that may be incorporated into moveable magazine component 22C and used with the multi-channel pipette device 160. The microwell 70C contains a fluid 170, such as a reagent, in a reservoir region 172. The microwell 70C further has an opening or inlet 174. While not shown, prior to the moveable magazine component 22C being used in the automated microfluidic reactor 20C, the inlet 174 of each microwell 70C may be sealed with a fluid-tight layer or film, as described above. Each individual microwell 70C may be sealed with individual separate removable seals or a larger removable seal may cover the entire surface of the moveable magazine component 22C. Thus, each reservoir region 172 is sealed, so that the seals are removed (by manual removal or puncturing) prior to measurement or testing. Notably, where the seal may be manually removed, no needle needs to be used to puncture the seal(s).

As shown, a single microfluidic connector 24C in the form of a cylindrical tube has the first aperture 152C. After the seal is removed, the microfluidic connector 24C can reversibly interface with the microwell 70C and when the first aperture 152A contacts the fluid 170, it can move the fluid into or out of the microfluidic connector 24C. For example, in some operational modes, the microfluidic connector 24C may draw the fluid 170 into the microfluidic connector 24C, including into the reactor chamber 40C. The multi-channel pipette device 160 can also draw a test sample (not shown) into the reactor chamber 40C either prior to or after drawing the fluid 170, so that the fluid 170 and a test sample are combined together for assessing any reaction and detecting any analytes present in the test sample.

FIG. 12B shows a second alternative embodiment of a single microwell 70C that may be incorporated into the moveable magazine component 22C and used with the multi-channel pipette device 160. To the extent that the features of the various components of the microwell and the microfluidic connector are the same as those of earlier variations shown in FIG. 12A, the same reference numbers are used herein and for brevity will not be described again in detail unless distinctions in the design are otherwise noted. As shown, a single microfluidic connector 24D in the form of a cylindrical tube has a needle 180 defining the first aperture 152D at a terminal end. The microfluidic connector 24D can reversibly interface with the microwell 70C. When the needle 180 having the first aperture 152D contacts the fluid 170, it can move the fluid into or out of the microfluidic connector 24D and thus into or out of the reactor chamber 40C in the at least one microfluidic connector 24D. As discussed above, the inlets 174 of the reservoirs 172 of each microwell 70C can thus be sealed and pierced individually and the needle 90 on the terminal end of the microfluidic connector 24D can serve to pierce through each reservoir seal on each microwell 70C.

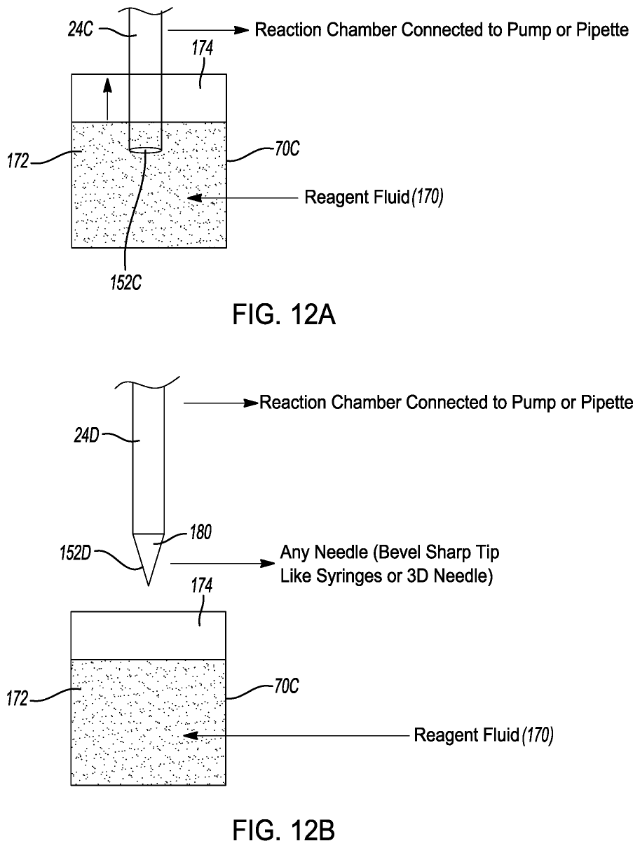

FIG. 13 shows a portion of another variation of an automated microfluidic reactor 20D, showing a moveable magazine component 22D in the form of a reservoir disk component having a plurality of microwells 70C defining a pattern of a plurality of rows or lines 182 extending radially from a center to a circumferential perimeter of the disk component/moveable magazine component 22D (e.g., in a spoke-like configuration). As shown, a first line 182A may contain a first reagent (Reagent 1), a second line 182B may contain a distinct second reagent (Reagent 2), and a third line 182C may contain a distinct third reagent (Reagent 3). As will be appreciated, the configuration of reagents shown in FIG. 13 is one example, but any variety of concentrations or distinct reagents may be present in various distinct microwells 70C in a given line or row. This variation of the automated microfluidic reactor 20D may be used with an associated pump (e.g., a syringe pump). Variations are contemplated where the microwell reservoirs of the moveable magazine component 22D are either at the bottom (underneath) or at the top of the microfluidic connectors 24C on the multi-channel component 150. This may be used with the microfluidic connector design shown in FIG. 12A, by way of example. In this case, the fluid 170, for example, each reagent (Reagents 1, 2, or 3) is pulled into the reaction chamber (not shown) via the microfluidic connector 24C, and after incubation, the contents are pulled into the disposable syringe pump, and the syringe may be replaced between the measurements.

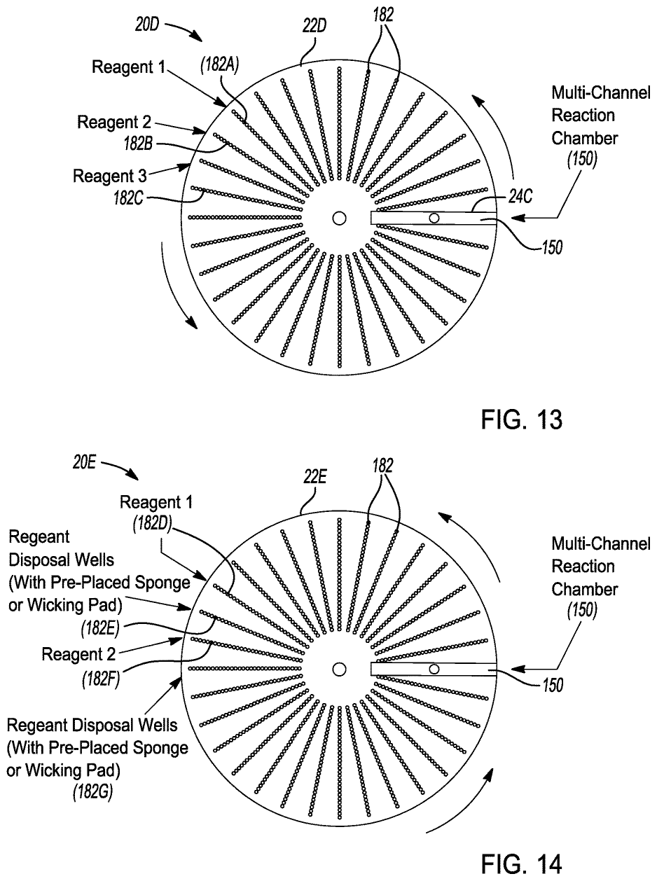

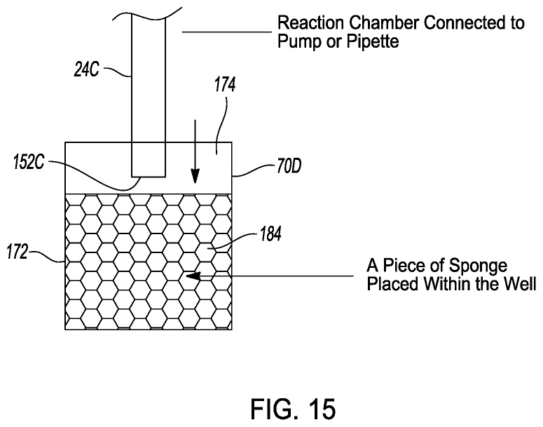

FIG. 14 shows another variation of portions of an automated microfluidic reactor 20E, showing a moveable magazine component 22E in the form of a reservoir disk component having a plurality of microwells defining a pattern of the plurality of rows or lines 182 extending radially from a center to a circumferential perimeter of the reservoir disk component 22E (e.g., in a spoke-like configuration). As shown, a first line 182D may contain a first reagent (Reagent 1) contained in reservoirs 172 of microwells 70C, a second line 182E may have a plurality of microwells 70D that contain a wicking or absorbent material 184 disposed in the reservoir region 172, as shown in FIG. 15. The wicking or absorbent material 184 may be formed of materials like sponges, fabrics, papers, felt, fibrous mats or pads and the like that are suitable for wicking and absorbing fluids, which may be deposited through the first aperture 152C of the microfluidic connector 54C. With renewed reference to FIG. 14, a third line 182F may contain a distinct second reagent (Reagent 2). A fourth line 182G may contain a wicking or absorbing material, as in FIG. 15. As will be appreciated, the configuration of reagents and wicking microwells shown in FIG. 14 is one example, but any variety of concentrations or distinct reagents and wicking microwells may be present. This embodiment is suitable for use with a configuration where the microfluidic connector 24C is disposed above the microwells 70C/reservoir regions 172 containing reagent and thus approaches them from top to bottom. In this variation, the reaction chamber 40C is integrated into/the same as microfluidic connectors 24C. For example, a reaction (or ELISA) takes places on a surface of the inner walls of the reaction chamber 40C (e.g., a hollow cylinder of any suitable material). This variation of the automated microfluidic reactor 20E could be used with an associated multichannel pipette. In operation, the reagents withdrawn from the microwells 70C (e.g., Reagent 1 or 2) from the first line 182D or the third line 182F is subsequently dispensed back into the next row of microwells 70D in the reservoir region 172 that contains the wicking or absorbent material 184, such as pre-placed wicking pads or sponge pieces to help collect and soak the reagent that is being dispensed back into the microwells 70D from the reaction chambers 40C in the microfluidic connector 26C after incubation.

Various embodiments of the inventive technology can be further understood by the specific examples contained herein. Specific Examples are provided for illustrative purposes of how to make and use the compositions, devices, and methods according to the present teachings and, unless explicitly stated otherwise, are not intended to be a representation that given embodiments of this invention have, or have not, been made or tested.

### Example 1

### Sandwich ELISA Protocol

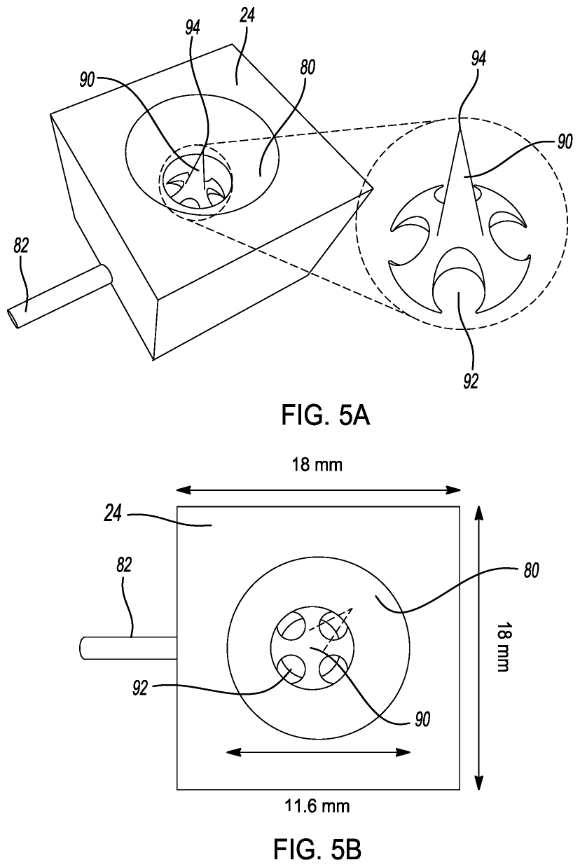

The process steps of the sandwich-ELISA protocol used here is shown in FIG. 6. It includes two parts, preparation and detection.

The preparation part involves the following steps. (1) Capture antibody incubation for 60 minutes, followed by washing. (2) 3% Blocker buffer bovine serum albumin (BSA) in 1× phosphate buffered saline (PBS) incubation for 30 minutes to block the unbound sites of the reaction chamber, followed by washing. (3) Superblock blocking buffer incubation for 15 minutes, followed by washing. The total preparation phase in this example takes about 105 minutes and is done before the detection part using the same machine and disposables (disks, microfluidic connector) described previously.

The detection part involves the following steps. (1) Sample incubation for 20 minutes, followed by washing. (2) Detection antibody incubation for 20 minutes, followed by washing. (3) Horseradish Peroxidase (HRP) enzyme incubation for 4 minutes, followed by washing. (4) Substrate addition and incubation for a few seconds, followed by chemiluminescence signal detection. The total time for the detection part is approximately 45 minutes, neglecting time for mechanically moving components and washing steps.

In this example, the following materials are used. A Human IL-6 DuoSet ELISA kit (DY206) and the 25× Wash Buffer Concentrate (WA126) are purchased from R&D Systems. The Blocker BSA (10% in PBS, 37525), the SuperBlock Blocking Buffer (37517), the Pierce Streptavidin Poly-HRP (21140), the Poly-HRP Dilution Buffer (N500), and the SuperSignal ELISA Femto Substrate (37074) are purchased from ThermoFisher Scientific. The Clear Resin V4.1 cartridge (RS-F2-GPCL-04) was purchased from Formlabs. Step motors, linear actuator, Arduino UNO microcontrollers, step motor drivers, peristaltic pump, and all other electrical parts were purchased from Amazon. The camera lens (FUHF125SA1) was purchased from B&H. The camera (MU530-BI-CK) was purchased from AmScope. Polystyrene capillaries were purchased from Optofluidic Bioassay LLC. See details of the mechanical and optical parts in Table 1.

### 3D Printing

The CAD design of the 3D printable parts was created using the Autodesk Fusion 360 software, and the models were intended for printing on a Formlabs 3B+3D printer, which uses Stereolithography (SLA) technology. A Clear Resin V4.1 was used for forming both the reservoir moveable magazine component and the microfluidic connector. The CAD designs were saved in the .stl format and the print orientation along with other settings was selected using Preform software, developed by Formlabs, to modify the .stl file.

The overall design was optimized to avoid delicate features, which allows for the use of the lowest 3D printing resolution (0.1 mm) and minimizes printing time. For the 3D printing resolution of the microfluidic connector, the “adaptive” option was chosen. This has the second highest printing speed after the 0.1 mm resolution among all resolution options, and its printing time was similar to the one using the 0.1 mm resolution. The reason to choose the adaptive resolution was to ensure that the slightly delicate features such as the bending narrow channel within the microfluidic connector were printed out properly. After testing different resolutions, it was observed that the 0.1 mm resolution could result in the possibility of narrow bending channels not being printed fully. Then the print file was uploaded to the printer using the Preform software.

The print platform area of the Formlabs 3B+3D printer has dimensions of 145×145 mm². The fastest prints are done when the structure's wider features are aligned horizontally, in other words, when the number of printable layers in the vertical direction is minimized. This would require the reservoir moveable magazine component to be placed laterally on the print platform in the Preform software, which results in not having enough space for another disk to be placed on the platform, since the diameter of the reservoir disk is 112 mm. Therefore, in the print platform of one printer, only one reservoir disk (with the current design) can be printed at a time. In this configuration, the print of each reservoir disk takes 3 hours. However, printing multiple similar objects simultaneously can significantly reduce the printing time per unit. Therefore, if a larger print platform (for example, UltiMaker 3D printer) is used, or if the dimensions of the reservoir moveable magazine component is reduced, a single platform can accommodate more reservoir disks, which would lead to a significant reduction in the per unit print time. Using the same platform of 3B+ Formlabs 3D printer that can fit 28 microfluidic connectors, it took approximately 280 minutes to complete the printing task.

A 3D-printed moveable magazine component is shown in FIGS. 3D and 3E. The thick disk was used to avoid potential bending during the UV curing as a part of post processing the 3D-printed parts. In this design, each disk has a number of reagent reservoir microwells that can cover three sandwich ELISA runs, which results in an economical cost per test (for example, estimated to be between $6 to $7 dollars/test). For a competitive ELISA test, each microwell/reservoir covers six tests, leading to an estimated cost per test of about $3 to $4. A 3D printed microfluidic connector is shown in FIG. 5D.

### Assembly and Operation of a Complete System

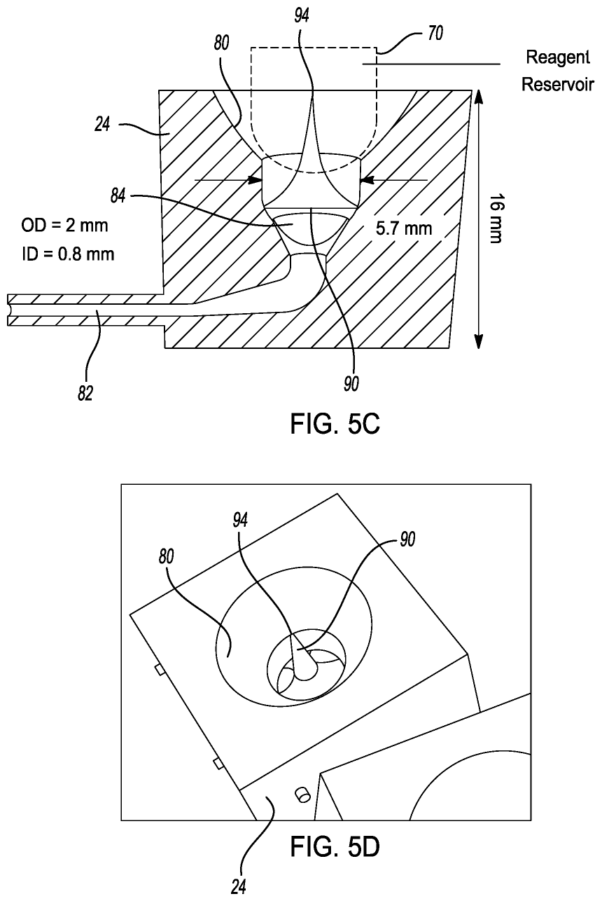

FIG. 7 shows the schematic of the full map of the biosensor device and how its components are assembled. As mentioned before, the rotational movement of the reservoir disk and the vertical movement of the microfluidic connector are the only mechanical movements required for the operation of the device. As shown in FIG. 7, the reservoir moveable magazine component is mounted on a step motor for its rotation, and the microfluidic connector is mounted on a linear actuator for its up/down movement.

Through a rotational movement of the step motor, the desired reagent reservoir/microwell moves to the top of the microfluidic connector. Then, through the upward movement of the vertical linear actuator, the microfluidic connector moves upward until its needle pierces through the aluminum seal at the bottom of the reagent reservoir, which releases the reagent fluid into the microfluidic connector's channel. Then, the pump (not shown) pulls the reagent into the system for a pre-determined duration and stops once the reagent arrives at the reaction chamber.

Once the specific incubation time of the reagent is over, the reagent/fluids are pulled out of the reaction chamber into the waste system, which can be the syringe itself, using commercially available pumps or custom-made syringe pumps. Then, the microfluidic connector moves downward to be disconnected from the reservoir, and the reservoir moveable magazine component rotates to the location of the wash buffer outlet (see e.g., outlet 136 of wash fluid/buffer channel 130 in inset of FIG. 3D). While a peristaltic pump is selected in this configuration to pump the wash buffer and move it from the wash buffer reservoir into the inlet above the reservoir disk/moveable magazine component, it will be appreciated that other types of pumps are likewise contemplated.

The wash buffer outlet 136 at the bottom of the moveable magazine component is internally connected to establish fluid communication with an inlet (see inlet 132) at the top the moveable magazine component (FIG. 3D, inset), which is connected to a large wash buffer reservoir (by way of non-limiting example, a 50 ml conical centrifuge tube here) via a peristaltic pump, as shown in FIG. 7. During rinsing, the peristaltic pump adds a few drops of the wash buffer into the microfluidic connector, until the tip of the needle is covered with the wash buffer (see FIG. 5C), and after a few seconds, it is pulled by the pump through the reaction chamber to the waste. The above process can be repeated multiple times until the desired amount of the wash buffer is used. The amount of wash buffer used in each of the wash steps was chosen to be a few hundred micro-liters to ensure a thorough wash of the reagents undesirably accumulated in the dead volumes and to provide a proper wash of the tip of the needle to rinse it. In the end, after a few seconds of wash buffer incubation inside the reaction chamber, the wash buffer is fully pulled out of the system by the pump and the reservoir moveable magazine component rotates to place the next reagent reservoir in the protocol on the top of the microfluidic connector.

A wash system thus comprises a washing fluid and a second pump in fluid communication with the microfluidic connector, as well as with the movable magazine component. The wash system further comprises a fluid reservoir. In certain aspects, the moveable magazine component further comprises at least one wash inlet and at least one wash outlet for transporting the washing fluid from the fluid reservoir to the microfluidic component. It will be appreciated however, that in alternative variations, the wash stream from the fluid reservoir may bypass the movable magazine component and be directly connected to the microfluidic connector.

As discussed above, operation of each of the pump(s) and motor(s) present in the system may be controlled by a controller.

The steps mentioned above are repeated until all the incubation steps are completed, as discussed in the context of FIG. 6. Finally, the substrate reagent is pulled into the reaction chamber, where it generates a chemiluminescent signal, which is captured by a detector (camera) placed above the reaction chamber (see FIG. 1). During the course of the experiment, a disposable syringe attached to the pump collects all the waste fluids.

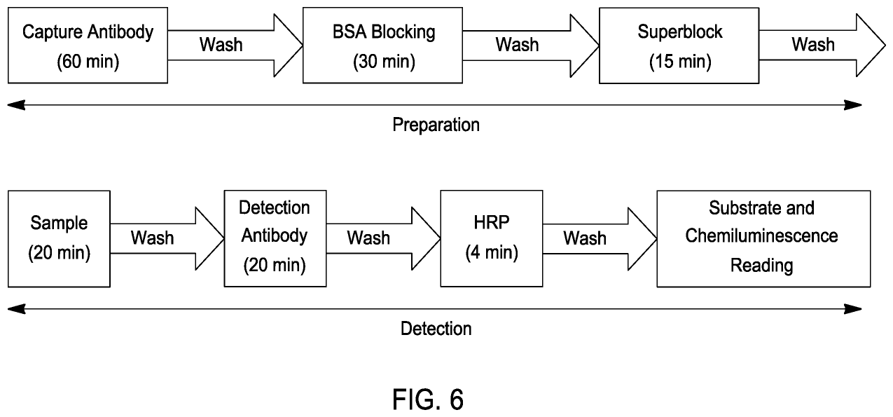

FIGS. 8A-8C show a completed and fabricated setup of an automated ELISA machine. Both the linear actuator and the step motor are driven by stepper motor drivers and are controlled using Arduino UNO microcontrollers. FIG. 8A is a 3D view of the complete setup. The dimensions of the area are 19 cm by 24 cm, and the highest part in the setup, which is the camera's position, is about 14 cm. The electronic parts, including the microcontrollers, drivers, and the bread board for voltage distribution are packaged on the corner of the setup shown in FIG. 7A. The side view photograph is shown in FIG. 7B. The small peristaltic pump used for pulling the wash buffer out of its reservoir and delivering it to the wash buffer inlet above the reservoir disk is also shown the in FIG. 7B. FIG. 7C shows a close-up photograph of the reaction chamber placed underneath the camera.

A total cost of the machine, including the linear stage, the step motor, peristaltic pump, microcontrollers, and motor drivers, is inexpensive and can be further reduced by industrial level optimizations and mass production/purchase of the parts. While commercially available syringe pumps are used in this example, alternative pumps can be formed with clamps, such as commercially available clamps or 3D printed clamps, and a linear actuator to form less expensive syringe pumps that also can be housed in the current device enclosure.

### Preparation of Disposables

For this variation of a sandwich ELISA device, reagents were prestored on each microwell, and the volume of the reagents can be less than 364 microliters (μL). Concentrations of each reagent used in pre-coating and detection IL-6 ELISA protocol are shown in Table 1.

TABLE 1

| | Concentration/volume used in | Container quantity | |
|---|---|---|---|
| Reagent | protocol | (conc.) | |
| Capture antibody | 10 μg/mL | 0.5 mL | |
| | (in 1X PBS) | (240 μg/mL) | |
| Detection antibody | 100 ng/mL | 1 mL | |
| | (in 3% BSA) | (3.00 μg/mL) | |
| BSA | 3% in 1X PBS | 600 mL | |
| | (~100 μL used) | (10% in 1X PBS) | |
| Superblock | No dilution | 1 | L |
| Poly-HRP | (0.05 μL used) | 0.5 | mL |
| Poly-HRP dilution | No dilution | 100 | mL |
| buffer | | | |
| Substrate | No dilution | 250 | mL |

| | | |
|---|---|---|
| Wash buffer | 1X diluted | 500 mL |
| | (~1 mL used) | (25X dilution) |
| Total reagents | — | — |

Many types of microfluidic reaction chambers can be used in accordance with the present disclosure. For simplicity, here, polystyrene capillary cartridges purchased from Optofluidic Bioassay LLC are used, which have been used previously in many ELISA applications. Each capillary cartridge comes with 12 individual capillaries (approximately 15 mm in length, 0.8 mm in inner diameter, about 2 mm in outer diameter, and about 8 mL in total chamber volume). One was used for each test. As the reaction chamber has only 8 mL inner volume, with much less than the approximate 364 microliters (μL) volume of the reagents used in the test, the entire chamber is filled with the reagents during the incubation. The capillary is connected to the outlet of the microfluidic connector upstream and to a pump downstream with flexible plastic tubing (as shown in FIG. 1).

### IL-6 Detection

Although the ELISA biosensor is compatible with various types of ELISA formats (such as direct ELISA, indirect ELISA, sandwich ELISA, and competitive ELISA), here sandwich ELISA is selected because it has the highest number of steps and the level of complexity in the detection process, to demonstrate the ability of our ELISA biosensor. Furthermore, interleukin-6 (IL-6) is used, which plays a key role in the immune response, particularly in inflammation and infection.

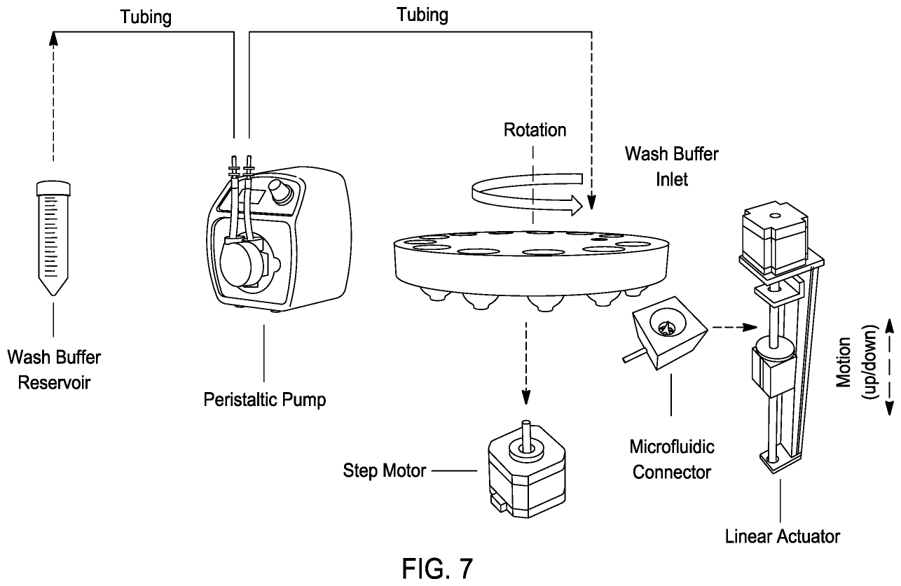

The tested concentrations included 0, 200, 400, 700, and 1000 pg/mL. The chemiluminescence images were captured. After putting the images together as shown in FIG. 9A, the channels are split into red, green, blue (RGB) pixels, and the blue channel is shown in FIG. 9B. The intensity profile was extracted using the ImageJ™ software based on the blue channel version of the image, and intensity count versus horizontal position is shown in FIG. 9C. The intensity count was extracted from the rectangular selection area shown in FIG. 9B, and it ranged from 0 to 255. The ImageJ software draws the intensity versus horizontal position, in the selected area, while taking the average in the vertical direction for each given horizontal point. To ensure that the intensity of the center of the capillary was selected for concentration readings, the middle point intensity is used in each capillary's intensity profile. FIG. 9D shows the calibration curve, i.e., intensity versus concentration for the abovementioned concentrations. The linear fitting shown in FIG. 9D shows a fit with an R² value of 0.96.

In this example, the details are provided for developing a miniaturized automated, and cost-effective ELISA device with a small size and light weight that can be used for POC applications and in the resource-limited regions.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. An automated microfluidic reactor for analyzing a test sample to be reacted with one or more reagents, the reactor comprising:
a moveable magazine component comprising a plurality of microwells, wherein each microwell of the plurality of microwells comprises an inlet and a reservoir, wherein at least one of the plurality of microwells is configured to hold one or more reagents;
at least one microfluidic connector comprising at least one microfluidic channel having an aperture, the at least one microfluidic connector reversibly interfacing with at least one microwell of the plurality of microwells to establish fluid communication therebetween;
a first motor for translating the moveable magazine component with respect to the microfluidic connector;
a second motor for translating the microfluidic connector from at least a first position where the microfluidic connector contacts the moveable magazine component to a second position where the microfluidic connector is not in contact with the moveable magazine component, wherein at least one of the translating the moveable magazine component or the translating of the at least one microfluidic connector is rotational and the other is linear;
at least one reactor chamber in fluid communication with the microfluidic channel of the microfluidic connector, wherein the at least one reactor chamber is configured to receive a test sample optionally comprising one or more target analytes; and
a pump or a pipette device for transporting fluids from the moveable magazine component through the microfluidic connector to the reactor chamber.

2. The automated microfluidic reactor of claim 1, wherein the translating of the moveable magazine component is rotating and the translating of the at least one microfluidic connector is linear.

3. The automated microfluidic reactor of claim 1, where the pump or the pipette device is for transporting fluids from the moveable magazine component through the microfluidic connector to the at least one reactor chamber.

4. The automated microfluidic reactor of claim 1, wherein the moveable magazine component has one or more covers or seals that may be removed or punctured to establish fluid communication with one or more of the plurality of microwells.

5. The automated microfluidic reactor of claim 1, wherein the at least one reactor chamber is a plurality of reactor chambers and the at least one microfluidic connector is a multi-channel microfluidic connector comprising a plurality of microfluidic channels in fluid communication with the plurality of reactor chambers.

6. The automated microfluidic reactor of claim 1, wherein at least one microwell of the plurality of microwells further comprises a wicking material.

7. The automated microfluidic reactor of claim 1, further comprising at least one detector for analyzing fluids in the at least one reactor chamber.

8. The automated microfluidic reactor of claim 7, further comprising a control system for controlling operation of the first motor, the second motor, the pump or the pipette device, and the at least one detector.

9. The automated microfluidic reactor of claim 1, wherein at least a portion of the microwells of the plurality of microwells store the one or more reagents.

10. The automated microfluidic reactor of claim 1, wherein the test sample comprises one or more target analytes and the one or more reagents are used in one or more reactions to detect the one or more target analytes.

11. The automated microfluidic reactor of claim 10, wherein the one or more reagents are part of an Enzyme Linked ImmunoSorbent Assay (ELISA)-based system.

12. The automated microfluidic reactor of claim 11, further comprising one or more detectors for measuring one or more of luminescence, fluorescence, or colorimetry to determine an amount of the one or more target analytes present in the test sample.

13. The automated microfluidic reactor of claim 12, further comprising at least one processor configured to receive output from one or more detectors and configured to determine an amount of the one or more target analytes present in the test sample.

14. The automated microfluidic reactor of claim 1, further comprising a plurality of microfluidic connectors, wherein each of the plurality of microfluidic connectors is configured to engage independent microwells of the plurality of microwells in the moveable magazine component, wherein each of the plurality of microfluidic connectors is in fluid communication with the at least one reactor chamber.

15. The automated microfluidic reactor of claim 14, wherein the at least one reactor chamber comprises a plurality of reactor chambers and each of the plurality of microfluidic connectors is in fluid communication with a distinct reactor chamber of the plurality of reactor chambers.

16. An automated microfluidic bioreactor for analyzing a sample to be reacted with one or more reagents, the bioreactor comprising:
a moveable magazine component comprising a plurality of microwells, wherein each microwell of the plurality of microwells comprises an inlet and a reservoir, wherein at least one of the plurality of microwells is configured to hold one or more reagents used in an enzyme-linked immunosorbent assay (ELISA) to detect one or more target biological analytes;
at least one microfluidic connector comprising at least one microfluidic channel having an aperture, the at least one microfluidic connector reversibly interfacing with at least one microwell of the plurality of microwells to establish fluid communication therebetween;
a first motor for translating the moveable magazine component with respect to the microfluidic connector;
a second motor for translating the microfluidic connector from at least a first position where the microfluidic connector contacts the moveable magazine component to a second position where the microfluidic connector is not in contact with the moveable magazine component, wherein at least one of the translating the moveable magazine component or the translating of the at least one microfluidic connector is rotational and the other is linear;
at least one reactor chamber in fluid communication with the microfluidic channel of the microfluidic connector, wherein the at least one reactor chamber is configured to receive a test sample optionally comprising one or more target biological analytes; and
a pump or a pipette device for transporting fluids from the moveable magazine component through the microfluidic connector to the reactor chamber.

17. The automated microfluidic bioreactor of claim 16, wherein at least a portion of the microwells of the plurality of microwells store the one or more reagents.

18. The automated microfluidic bioreactor of claim 16, wherein the at least one reactor chamber comprises a capture antibody capable of binding with at least one of the one or more target biological analytes and a detection antibody that comprises an indicator species.

19. The automated microfluidic bioreactor of claim 18, wherein the one or more reagents includes a substrate, wherein a third microwell of the plurality of microwells comprises the substrate.

20. The automated microfluidic bioreactor of claim 16, further comprising one or more detectors for measuring one or more of luminescence, fluorescence, or colorimetry to determine an amount of the one or more target biological analytes present in the test sample in the at least one reactor chamber.
